SEQUENCE LISTING (6 sequences)

SEQ ID NO: 1 - protein, 115 aa, Homo sapiens
  MIF PROTEIN HUMAN (Accession# CAG30406) (1)..(115)
MPMFIVNTNVPRASVPDGFLSELTQQLAQATGKPPQYIAVHVVPDQLMAFGGSSEPCALCSLHSIGKIGGAQNRSYSKLLCGLLAERLRISPDRVYINYYDMNAANVGWNNSTFA

SEQ ID NO: 2 - DNA, 348 bp, Homo sapiens
  MIF DNA (Accession# CR456520) (1)..(348)
atgccgatgttcatcgtaaacaccaacgtgccccgcgcctccgtgccggacgggttcctctccgagctcacccagcagctggcgcaggccaccggcaagcccccccagtacatcgcggtgcacgtggtcccggaccagctcatggccttcggcggctccagcgagccgtgcgcgctctgcagcctgcacagcatcggcaagatcggcggcgcgcagaaccgctcctacagcaagctgctgtgcggcctgctggccgagcgcctgcgcatcagcccggacagggtctacatcaactattacgacatgaacgcggccaatgtgggctggaacaactccaccttcgcctaa

SEQ ID NO: 3 - protein, 742 aa, Homo sapiens
  CD44 Protein (Accession # NP_000601) (1)..(742)
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 4 - DNA, 2229 bp, Homo sapiens
  CD44 human (Accession # NM_000610) (1)..(2229)
atggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaa

SEQ ID NO: 5 - protein, 296 aa, Homo sapiens
  CD74 human (Accession # NP_001020330) (1)..(296)
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKVLTKCQEEVSHIPAVHPGSFRPKCDENGNYLPLQCYGSIGYCWCVFPNGTEVPNTRSRGHHNCSESLELEDPSSGLGVTKQDLGPVPM

SEQ ID NO: 6 - DNA, 891 bp, Homo sapiens
  CD74 human (Accession # NM_001025159) (1)..(891)
atgcacaggaggagaagcaggagctgtcgggaagatcagaagccagtcatggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagaatgctgaccccctgaaggtgtacccgccactgaaggggagcttcccggagaacctgagacaccttaagaacaccatggagaccatagactggaaggtctttgagagctggatgcaccattggctcctgtttgaaatgagcaggcactccttggagcaaaagcccactgacgctccaccgaaagtactgaccaagtgccaggaagaggtcagccacatccctgctgtccacccgggttcattcaggcccaagtgcgacgagaacggcaactatctgccactccagtgctatgggagcatcggctactgctggtgtgtcttccccaacggcacggaggtccccaacaccagaagccgcgggcaccataactgcagtgagtcactggaactggaggacccgtcttctgggctgggtgtgaccaagcaggatctgggcccagtccccatgtga

(19) United States
(12) Patent Application Publication
(10) Pub. No.: US 2008/0089884 A1
(43) Pub. Date: Apr. 17, 2008

(54) Compositions and methods for the modulation of detrusor activity

(76) Inventors: George A. Kuchel, Avon, CT (US); Richard Bucala, New Haven, CT (US)

Correspondence Address: MCCARTER & ENGLISH LLP;CITYPLACE I, 185 ASYLUM STREET, HARTFORD, CT, 06103, US

(21) Appl. No.: 11/825,253

(22) Filed: Jul. 3, 2007

Related U.S. Application Data
(60) Provisional application No. 60/818,721, filed on Jul. 6, 2006.

(51) Int. Cl. A61K 31/42; A61K 31/70; A61K 38/02; A61K 39/395; A61P 13/00; C07H 21/02; C12Q 1/68

(52) U.S. Cl. 424/130.1; 514/2; 514/378; 514/43; 536/24.1

(57) ABSTRACT

The invention relates generally to methods for treating and/or preventing bladder disorders. In certain embodiments the invention comprises methods for the treatment and/or prevention of a bladder disorder comprising administering an effective amount of a therapeutic agent that decreases the expression, release or biological activity of macrophage migration inhibition factor (MIF) to a subject in need thereof. In other aspects the invention relates to a method for diagnosing bladder disease and/or bladder disease severity comprising screening for a MIF gene or MIF receptor gene polymorphism or expression level.

### INCORPORATION BY REFERENCE

In compliance with 37 C.F.R. § 1.52(e)(5), the sequence information contained on compact disc, file name: 98121.00131SEQLIST.ST25; created on: Jul. 5, 2006; size: 16 KB; and filed Jul. 6, 2006 in association with U.S. Provisional Patent Application No. 60/818,721 is hereby incorporated by reference in its entirety. The Sequence Listing information recorded in computer readable form (CRF), the written Sequence Listing provided herewith, and the Sequence Listing filed with U.S. Provisional Patent Application 60/818,721 are all identical with one another.

### FIELD OF THE INVENTION

The invention relates generally to therapeutic compositions and methods for treating and/or preventing pathologies related to improper detrusor activity.

### BACKGROUND

Both primary care providers and specialists increasingly manage voiding problems in the elderly. For example, in urology, older men account for nearly 50% of office visits and 62% of operations. Urinary incontinence and related lower urinary tract (LUT) symptoms impact geriatric health and independence. In particular, detrusor underactivity (DU) has received surprisingly little clinical and research attention. Nevertheless, this condition cannot be ignored since it can influence the clinical presentation and may impede the therapy of LUT disorders, as common and disparate as, detrusor over activity, acute or chronic urinary retention, and benign prostatic hyperplasia (BPH).

Detrusor underactivity (DU) has been defined as a contraction of reduced strength and/or duration, resulting in prolonged bladder emptying and/or a failure to achieve complete bladder emptying within a normal time span. European and US research groups have shown that many aspects of detrusor performance decline with old age, in many older individuals progressing to frank DU. “Normative” aging contributes to this process, since more modest declines in detrusor contractility can also be demonstrated in healthy older adults who are otherwise urodynamically normal. The presence of detrusor over activity and DU in the same individual which has been termed Detrusor Hyperactivity with Impaired Contractility (DHIC) by Resnick et al. possess particular challenges. Half or more of all nursing home residents are incontinent, with DHIC the most common urodynamic pattern. Management of these individuals remains unsatisfactory since anticholinergics may worsen retention, while no effective pharmacotherapy for DU currently exists.

Currently available treatments for DU are purely palliative if not wholly ineffective. Individuals in severe urinary retention require the placement of a urinary catheter. While this procedure is very common, it is associated with emotional and physical discomfort, as well as an increased risk of infection, sepsis and decreased mobility. Individuals with urinary tract infection complicated by urinary retention often undergo treatment with multiple courses of antibiotics since their infection may be very difficult to eradicate.

Therefore, there is an overwhelming need for therapeutics that treat and/or prevent DU and its related pathologies, which are both efficacious and well tolerated.

### SUMMARY OF THE INVENTION

Detrusor underactivity (DU) is a common geriatric condition that impairs the bladder's ability to empty. Its presence predisposes older adults to important complications such as urinary retention, renal failure and recurrent urinary tract infections. Nevertheless, the pathogenesis of detrusor underactivity remains unknown and care is palliative since no effective treatment is available. The present invention relates to the discovery that macrophage migration inhibitory factor (MIF) is a key factor in the pathogenesis of DU, and inhibition of its expression and/or activity can prevent the development of the two hallmarks of DU, bladder detrusor muscle cell death and collagen deposition.

In certain aspects the invention relates to compositions which inhibit the expression, release and/or biological activity of migration inhibitory factor (MIF). The invention further relates to the uses of such compositions and methods for the prevention and/or treatment of bladder disorders, for example, DU, DHIC, urinary retention, renal failure, and/or recurrent urinary tract infections which result from DU.

The invention is illustrated by working examples which demonstrate that MIF exacerbates bladder disorders such as DU, DHIC, urinary retention, renal failure, and/or recurrent urinary tract infections which result from DU. Other features and advantages of the invention will be apparent from the following description of the preferred embodiments thereof, and from the claims.

### BRIEF DESCRIPTION OF THE DRAWINGS

FIG. 1: Lower Urinary Tract Pathways Controlling Detrusor Contraction and Relaxation. Mechanisms involved in mediating bladder contraction and relaxation have been the subject of recent reviews. Following its release from parasympathetic nerve fibers, acetylcholine interacts with M3 muscarinic receptors to activate phospholipase C which generates inositol triphosphate (IP3). IP3 mediates calcium release from its stores in endoplasmic reticulum. Intracellular free calcium binds to calmodulin, which then activates the enzyme Myosin Light Chain Kinase (MLCK). MLCK mediates an ATP-dependent phosphorylation and activation of contractile muscle proteins triggering a detrusor muscle contraction. Activation of M2 muscarinic receptors may promote detrusor contractions by inhibiting (stippled line) adenylate cyclase activity which results in lower intracellular levels of a putative bladder relaxant, cyclic adenosine monophosphate (AMP). Stimulation of 3-adrenergic receptors by adrenaline released from sympathetic nerves relaxes bladder smooth muscle via adenylate cyclase activation. The cytoplasmic entry of extracellular calcium is promoted by ATP release from a variety of nerves and its interaction with purinergic receptors (e.g. P2X₁). Anticholinergic agents may pharmacologically block normal detrusor contractions through their interactions with M3 and M2 receptors. Caveolae are submembrane vesicles which represent additional sites of calcium flux generation. Muscarinic and purinergic receptors are among the signaling molecules known to cluster in caveolae.

FIG. 2: Potential vicious cycle of urinary tract infection, inflammation, detrusor underactivity and urinary retention. Majority of symptomatic urinary tract infections are caused by gram negative bacteria. In animal models, the intraluminal administration of LPS (Lipopolysaccharide), the major component of gram-negative bacterial cell wall, leads to inflammatory bladder changes involving c-fos, cyclooxygenase-2 and MIF (macrophage migration inhibitory factor). Moreover, bacterial cystitis, defined as the presence of a positive culture (>10⁵ cfu/ml) with presence of nitrites and leucocytes, was associated with a nearly 4-fold increase in urinary MIF levels (normalized to creatinine). MIF is a pro-inflammatory cytokine which is released from abundant urothelial stores by inflammatory and infectious stimuli. In genetically-modified mice, MIF has been shown to be implicated in the development of bladder muscle loss and fibrosis which develop in the setting of both chronic urinary retention and ovariectomy. Bladder muscle degeneration, fibrosis and axonal degeneration represent the major features of detrusor underactivity in human biopsies from older adults with this condition. Detrusor underactivity may enhance the risk of urinary retention, impeding the treatment of urinary tract infection. These relationships could result in the development of a vicious cycle with chronic infection and inflammation promoting greater DU and urinary retention which in turn could make urinary tract infection more likely and more difficult to eradicate. Effects of estrogen depletion on bladder inflammation, muscle loss and fibrosis may be in mediated, at least in part via MIF, since estrogen inhibits inflammation-mediated release of MIF from macrophages. It remains to be seen what extent the apparently beneficial effects of intravaginal estrogen on recurrent infections in post-menopausal women are mediated via these mechanisms.

FIG. 3: Identifying a downstream signaling pathway shared by more than one risk factor for detrusor underactivity. Multiple clinical risk factors may contribute to the development of detrusor underactivity (Table 1). The multifactorial nature of such common geriatric syndromes has posed obstacles to the conduct of research which is mechanistic rather than descriptive. Nevertheless, the clinical observation that diverse risk factors may behave synergistically suggests the presence of some common downstream pathways in the pathogenesis of such complex conditions. Bladder muscle degeneration, fibrosis and axonal degeneration represent the major features of detrusor underactivity in human biopsies from older adults with this condition. Recent studies conducted in genetically-modified mice, have implicated MIF, a pro-inflammatory urothelium-derived cytokine, in the development of bladder muscle loss and fibrosis which develop in the setting of chronic urinary retention and after ovariectomy (regular lines).

FIG. 4: Images of MIF immunoreactivity in urothelial cells in wild-type female mice, three weeks after sham (A) or partial bladder outlet obstruction (pBOO) surgery (B). MIF immunoreactivity was highly intense in all urothelial cell layers in sham-operated wildtype animals (A). In response to pBOO surgery, urothelium (dark regions) appeared thinner with significantly decreased MIF immunoreactivity. In bladders from MIF knockout (K/O) animals, no MIF immunoreactivity was detected (not shown). Calibration bar=40 μm.

FIG. 5: Changes in COX-2 (A) and MIF (B) mRNA following sham or partial bladder outlet obstruction (pBOO) surgery. Analysis was by quantitative PCR. RQ values were calculated using the ct method. Each bar reflects the mean +SEM of 4-6 animals. *=significantly different from sham control animals, p<0.05.

FIG. 6: Effect of partial bladder outlet obstruction and MIF on appearance of trichrome stained detrusor sections. Low magnification images were obtained from WT (A, B) and MIF K/O (C, D) mice 3 weeks after sham (A, C) or pBOO (B, D) surgery. Calibration bar=1 mm. Higher magnification images were also obtained from WT (a, b) and MIF K/O (c, d) mice 3 weeks after sham (a, c) or pBOO (b, d) surgery. Calibration bar=0.2 mm.

FIG. 7: Cellular phenotype of cultured bladder cells. A. Following trypsinization, rat bladder muscle culture cells were immunolabeled using three smooth muscle (a-smooth muscle actin, myosin light chain kinase and myosin heavy chain) and one epithelial (cytokeratin-17) markers. They were then resolved using flow cytometry. Nearly all (99.7%) of cultured cells expressed α-smooth muscle actin. Other smooth muscle markers were also common, with most (80%) cells expressing myosin light chain kinase and 26% expressing myosin heavy chain. Cytokeratin-17, a urothelial marker, was not detected in our cultures. These well-differentiated bladder muscle cells released MIF protein after being exposed to TNF-α (50 ng/ml) over 24 hours. MIF was measured using ELISA in supernatants obtained from 3 separate cultures (*; p<0.01).

FIG. 8: Primary rat bladder muscle cultures express α-smooth muscle actin and make contact via cadherin-positive junctions. Most cells in our primary bladder muscle cultures expressed α-smooth muscle actin (seen as rod-like fibers). Confocal microscopy revealed areas of cadherin-positive contact (arrows) with a “zipper”-like appearance previously described with epithelial cells.

FIG. 9: MIF Effect on TUNEL staining. Primary rat detrusor muscle cultures were processed for TUNEL staining after a 3 hour incubation in the presence (B) or absence of (A) rMIF protein (100 ng/ml). A minimum of 100 cells were counted in each of 6 separate slides. The percentage of TUNEL-positive slides increased from 7.8±2.2 to 20.0±3.9 (p<0.05).

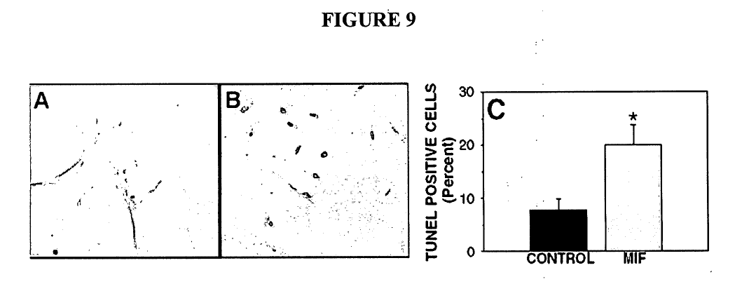

FIG. 10: Annexin V Labeling and PI Uptake in MIF-treated Cultures. Primary rat detrusor muscle cultures were studied in the absence or presence of rMIF protein (100 ng/ml) for 24 hrs. Floating and trypsinized adherent cells were labeled with Annexin V (An) and Propidium Iodide (PI) to separate early apoptotic, late apoptotic and healthy cells using flow cytometry. In three independent experiments, MIF increased the proportion of early (4.2±0.7 vs 23.9±6.3;*) and late (7.3±1.1 vs 31.1±5.2;*) apoptotic cells, while decreasing healthy (85.6±1.3 vs 42.5±8.6;*) cells (*; p<0.001).

### DETAILED DESCRIPTION OF THE INVENTION

The present invention is based on the discovery that the biological activity of MIF plays a key role in the progression of bladder disorders such as detrusor underactivity (DU), and detrusor hyperactivity (DHIC). Detrusor muscle loss and collagen deposition represent two morphologic features which are common and central to both detrusor decompensation in bladder outlet obstruction (BOO) and idiopathic DU. Such structural changes can be measured precisely, and in humans may, in fact, precede the development of urodynamically evident DU.

Currently, the clinician has to rely largely on anecdotal personal clinical experiences when evaluating the safety of specific medications in individuals with DU. Finally, as noted above, the management of urgency and incontinence in older adults who have both detrusor over activity and DU is highly unsatisfactory. There is an important need to evaluate the efficacy and safety of currently used antispasmodics in such individuals, including those who have severe DU. Non-pharmacologic approaches to incontinence management are often effective in many older adults and should also be tested in individuals unable to tolerate traditional antispasmodics, including the frail elderly with DU. Above all, it is no longer possible to extrapolate data from research conducted in other populations to the frail elderly. There is an urgent need to move beyond conceptual and pharmacological approaches to urinary incontinence which date to the 19^{th} century and to begin integrating more recent technological developments into the care of the frail elderly.

As such, the present invention relates to compositions and methods that inhibit MIF expression, release, and/or activity in vivo, for the treatment and/or prevention of bladder disorders, for example, DU; DHIC; urinary retention; renal failure; and/or recurrent urinary tract infections which result from DU. The invention further discloses therapeutic, diagnostic, and research methods for diagnosis, treatment, and prevention of disorders involving MIF dysregulation.

The inhibition of MIF activity in accordance with the invention may be accomplished in a number of ways, which may include, but are not limited to, the use of factors which bind to MIF and neutralize its biological activity (e.g., binding proteins, antibodies and/or antibody fragments); the use of MIF-receptor (e.g., CD44 and CD74) antagonists; the use of compounds that inhibit the release of MIF from cellular sources in the body; the use of antagonist compounds that bind directly to MIF (e.g., isoxazolines); the use of nucleotide sequences derived from MIF coding, non-coding, and/or regulatory sequences to prevent or reduce MIF expression (e.g., small inhibitory RNAs, shRNA, miRNA, antisense RNAs, and the like); and the use of nucleotide sequences derived from MIF-receptor coding, non-coding, and/or regulatory sequences to prevent or reduce MIF expression. Any of the foregoing may be utilized individually or in combination to inhibit MIF activity in the treatment of the bladder disorders, and further, may be combined with any other suitable therapeutic, for example, steroids, anti-inflammatory, antibiotics, bethanechol, or any combination thereof.

As used herein, the term “MIF antagonist” or “antagonist of MIF” is used generally to refer to an agent capable of direct or indirect inhibition of MIF expression, release, and/or activity; and includes antagonists of MIF, MIF binding proteins, and MIF receptors. Also, as used herein “MIF receptor” relates generally to any protein or fragment thereof capable of undergoing binding to a MIF protein.

As used herein, “bladder disorder” is used broadly and encompasses, for example, DU, DHIC, urinary retention, renal failure, and/or recurrent urinary tract infections which result from DU.

In certain aspects, the inhibition of MIF activity is accomplished by, for example, the use of MIF binding partners, i.e., factors that bind to MIF and neutralize its biological activity, such as neutralizing anti-MIF antibodies, soluble MIF receptors (e.g., CD74 and/or CD44), MIF receptor fragments, and MIF receptor analogs; the use of MIF-receptor antagonists, such as anti-MIF-receptor antibodies, inactive MIF analogs that bind but do not activate the MIF-receptor, small molecules that inhibit MIF release, or alter the normal configuration of MIF, or inhibit productive MIF/MIF-receptor binding; or the use of nucleotide sequences derived from MIF gene and/or MIF receptor gene, including coding, non-coding, and/or regulatory sequences to prevent or reduce MIF expression by, for example, antisense, ribozyme, and/or triple helix approaches.

Antibodies to MIF proteins, MIF binding proteins and MIF receptors are presented in detail in U.S. Pat. No. 6,645,493, which is incorporated by reference in its entirety for all purposes. Any of the foregoing methods may be utilized individually or in combination to inhibit MIF release and/or activity in the treatment of the relevant conditions. Further, such treatment(s) may be combined with other therapies that inhibit or antagonize MIF activity or exhibit analgesic and/or anti-inflammatory activity.

In certain aspects the invention comprises methods for treating a bladder disorder comprising administering an effective amount of an MIF antagonist, for example, an antibody, an isoxazoline or a combination thereof. A detailed discussion of isoxazolines encompassed by the present invention, and methods for the preparation of isoxazolines is found in the following U.S. Patents and Published Patent Applications: U.S. Pat. No. 6,599,938 to Yousef Abed; US20030008908A1 to Yousef Abed; and US20040204464A1 to Yousef Abed, the teachings of which are incorporated herein by reference in their entirety for all purposes. In one embodiment, the invention relates to administering an effective amount of the isoxazoline, “ISO-1.”

In another aspect, the present invention features a nucleic acid molecule, such as a decoy RNA, dsRNA, siRNA, shRNA, micro RNA, aptamers, antisense nucleic acid molecules, which down regulates expression of a sequence encoding MIF or a MIF receptor, for example, CD44 and CD74. In an embodiment, a nucleic acid molecule of the invention is adapted to treat bladder disorders, for example, DU, DHIC, urinary retention, renal failure, and/or recurrent urinary tract infections which result from DU. In another embodiment, a nucleic acid molecule of the invention has an endonuclease activity or is a component of a nuclease complex, and cleaves RNA having a MIF, CD44 or CD74 nucleic acid sequence.

In one embodiment, a nucleic acid molecule of the invention comprises between 12 and 100 bases complementary to RNA having a MIF, CD44 or CD74 nucleic acid sequence. In another embodiment, a nucleic acid molecule of the invention comprises between 14 and 24 bases complementary to RNA having a MIF, CD44 or CD74 nucleic acid sequence. In any embodiment described herein, the nucleic acid molecule can be synthesized chemically according to methods well known in the art.

In another aspect the present invention provides a kit comprising a suitable container, the active agent capable of inhibiting MIF activity, expression or binding in a pharmaceutically acceptable form disposed therein, and instructions for its use.

In another aspect, the invention relates to a method for diagnosing or monitoring bladder disorder disease or progression comprising detecting for the presence of a nucleotide polymorphism in the MIF structural gene associated with bladder disease or disease severity, through the detection of the expression level of a MIF gene or protein or both. MIF polymorphisms have been identified that correlate with arthritis severity, lupus, and inflammation. (See, Zhong et al., Simultaneous detection of microsatellite repeats and SNPs in the macrophage migration inhibitory factor (MIF) gene by thin-film biosensor chips and application to rural field studies. Nucleic Acids Res. 2005 Aug. 2; 33(13):e121; Donn et al., A functional promoter haplotype of macrophage migration inhibitory factor is linked and associated with juvenile idiopathic arthritis. Arthritis Rheum. 2004 May; 50(5):1604-10; Sanchez et al, Evidence of association of macrophage migration inhibitory factor gene polymorphisms with systemic lupus erythematosus. Genes Immun. 2006 May 18; Lehmann et al., A single nucleotide polymorphism of macrophage migration inhibitory factor is related to inflammatory response in coronary bypass surgery using cardiopulmonary bypass. Eur J Cardiothorac Surg. 2006 July; 30(1):59-63. Epub 2006 Mar. 9.; Worthington J., Investigating the genetic basis of susceptibility to rheumatoid arthritis. J Autoimmun. 2005; 25 Suppl:16-20. Epub 2005 Oct. 27. Review; Sakaue et al., Promoter polymorphism in the macrophage migration inhibitory factor gene is associated with obesity. Int J Obes (Lond). 2006 February; 30(2):238-42; Nohara et al., Association of the −173 G/C polymorphism of the macrophage migration inhibitory factor gene with ulcerative colitis. J Gastroenterol. 2004; 39(3):242-6; Baugh et al., A functional promoter polymorphism in the macrophage migration inhibitory factor (MIF) gene associated with disease severity in rheumatoid arthritis. Genes Immun. 2002 May; 3(3):170-6; all of which are incorporated herein by reference in their entirety for all purposes.). As used herein, “MIF gene” or “MIF structural gene” includes the 5′ UTR, 3′ UTR, promoter sequences, enhancer sequences, intronic and exonic DNA of the MIF gene as well as the MIF gene mRNA or cDNA sequence.

As one of ordinary skill will comprehend, the MIF gene polymorphisms associated with bladder disorders and hence useful as diagnostic markers according to the methods of the invention may appear in any of the previously named nucleic acid regions. Techniques for the identification and monitoring of polymorphisms are known in the art and are discussed in detail in U.S. Pat. No. 6,905,827 to Wohlgemuth, which is incorporated herein by reference in its entirety for all purposes.

Certain aspects of the invention encompass methods of detecting gene expression or polymorphisms with one or more DNA molecules wherein the one or more DNA molecules has a nucleotide sequence which detects expression of a gene corresponding to the oligonucleotides depicted in the Sequence Listing. In one format, the oligonucleotide detects expression of a gene that is differentially expressed. The gene expression system may be a candidate library, a diagnostic agent, a diagnostic oligonucleotide set or a diagnostic probe set. The DNA molecules may be genomic DNA, RNA, protein nucleic acid (PNA), cDNA or synthetic oligonucleotides. Following the procedures taught herein, one can identify sequences of interest for analyzing gene expression or polymorphisms. Such sequences may be predictive of a disease state.

Clinical diagnosis of DU is problematic since symptoms are non-specific and the condition is defined urodynamically. This is especially problematic when DU leads to partial or chronic urinary retention. In this context, DU can contribute to other LUT symptoms such as urinary frequency and nocturia because of incomplete emptying. Moreover, as discussed below, DU can also confound the diagnosis of an overactive bladder. As a result of all these considerations, DU is a very common, yet highly under diagnosed geriatric condition.

Epidemiologic and Diagnostic Issues

Obstacles to clinical diagnosis and epidemiologic studies. Clinical diagnosis of DU presents major challenges for both clinicians and epidemiologists. Obstructive symptoms (hesitancy, weakened stream, intermittency, straining, sense of incomplete emptying) do not reliably predict bladder outlet obstruction (BOO), DU or even elevated post-void residuals (PVRs). Urodynamic studies have been used to define the presence of DU, yet it is not realistic to incorporate such a physiologic “gold standard” into the design of larger population-based studies. Since no practical proxy measure has yet been validated, major lacunes exist in our understanding of the natural history of DU.

In male retention many other key issues also remain unresolved. Over a half of men over 50 have LUT symptoms and BOO has been the main, often only, focus of efforts to address male retention. However, recent re-evaluations have demonstrated that urinary retention may occur in the absence of BOO and that co-existing morbidity such as DU may complicate the clinical presentation of BOO. Thus, detrusor decompensation may follow from prolonged untreated BOO. It has even been suggested that prostate surgery may be of little benefit to older men in the absence of significant BOO. The long-term impact of “watchful waiting” on detrusor performance remains unknown as does the question of why only some, but not other men with BPH develop DU. Finally, in older men, DHIC may present with “obstructive” symptoms in the absence of obstruction, requiring care and vigilance when diagnosing such individuals.

BOO is rare in older women, particularly in the absence of previous peri-urethral surgery. Nevertheless, 2.7% of women from a general population referred for voiding studies and 4% of incontinent female nursing home residents demonstrated evidence of obstruction. Nevertheless, in spite of a low risk of BOO, many groups of older women are at high risk for partial or complete urinary retention, suggesting a much higher than expected prevalence of DU in this population. This conclusion has been substantiated in the long-term care setting where DU is very common among incontinent nursing home residents, a highly vulnerable population which is approximately 80% female. As discussed below, women who have suffered a fracture of the hip or have undergone large joint orthopedic surgery are also at an extremely high risk of acute urinary retention. Many risk factors may contribute to DU (Table 1).

TABLE 1
Potential Risk Factors for Detrusor Underactivity

| Condition | Proposed Mechanisms Leading to Detrusor Underactivity |
|---|---|
| Acute | Complete or partial BOO leads to muscle fiber damage and decreased responsiveness to |
| Urinary Retention | pharmacological stimuli for contraction. Bladder decompression may promote healing and |
| | enhance likelihood of successful voiding trial. |
| Benign Prostatic | An early compensatory phase involves hypertrophy of individual muscle cells. This is |
| Hyperplasia (BPH) | followed by a decompensation phase with loss of detrusor muscle cells, collagen deposition |
| | and axonal degeneration. |
| Sacral Neuropathy | Sacral neuropathy may involve parasympathetic nerve fibers required for detrusor |
| | contraction. |
| Diabetes Mellitus | Prolonged diabetes mellitus may lead to axonal and muscle degeneration in the detrusor. |
| Aging | The dense-band pattern of normative human bladder aging could indicate decreased calcium |
| | signaling as a result of fewer caveolae. In animal models, aged bladders are unable to |
| | maintain raised pressure in response to BOO. |
| Menopause | In animal models, ovariectomy results in caveolar depletion, loss of contractile proteins, |
| | axonal degeneration and loss of muscle cells. |
| Fecal Impaction | Fecal impaction may promote obstruction via a direct mechanical effect. Animal and human |
| | studies suggest that anal distension diminishes detrusor contractility. |
| Urinary Tract | Bladder infectious stimuli induce inflammatory molecules, especially MIF (macrophage |
| Infection | migration inhibitory factor) which mediates muscle loss and fibrosis in urinary retention and |
| | after ovariectomy. |
| Anticholinergic | Anticholinergics, including over-the-counter (e.g. antihistamines) and nebulized ipratropium |
| Medications | bromide may compete with acetylcholine at muscarinic receptors. |
| Immobility | Immobile individuals are more likely to be in urinary retention, but co-morbid factors (e.g. |
| | constipation, medications) likely contribute. Retention does not appear to improve as older |
| | adults in inpatient rehabilitation recover mobility. |
| Anesthesia | Type of surgery, age, co-morbidity, and medications are far more important risk factors for |
| | post-operative retention than type of anesthesia |
| Surgery | Retention may occur after any surgery, yet hip fracture repair and total joint (hip or knee) |
| | arthroplasty carry a particularly high risk, while the risk associated with hernia repair, anal |
| | surgery and some gynecological procedures is intermediate. |
| Hip Fracture | Hip fractures represent a major risk factor for urinary retention. Contributions from |
| | immobility, constipation, local trauma, pain or shared pathophysiologic mechanisms (e.g. |
| | estrogen lack) remain to be defined. |
| Neuroleptics | Individual case reports of urinary retention induced by combinations of potent psychoactive |
| | drugs (e.g. fluoxetine-risperidone) |
| Calcium Channel | Calcium channel antagonists increase the odds ratio for acute urinary retention in older |
| Antagonists | men, yet relationship to DU is unclear. |
| Alpha Agonists | Sympathomimetic recreational drugs have been described as causes of retention in younger |
| | individuals. Similar medications should be discontinued in the setting of acute or worsening |
| | urinary retention. |

Detrusor contractility and incontinence. Bladder emptying results from a balance between two factors—detrusor muscle contraction and bladder outlet resistance. Bladder emptying and detrusor contractility are lower in old age. Maximal detrusor contraction strength, detrusor contractility in the absence of overt neurological disease or diabetes and detrusor contractility measured in healthy, continent and unobstructed men all decline with advanced age. Detrusor contraction strength was also lower in older women, as compared to middle-aged and younger controls.

Incontinence is a common and debilitating geriatric condition. US costs are estimated at well over $30 billion/year. DU and incontinence are especially prevalent in the frail elderly. Assessments must be global, since multiple risk factors and non-urologic morbidities may contribute. In a urodynamic study of 94 incontinent nursing home residents DHIC was the most common urodynamic finding, occurring in one-third. DU represents the impaired contractility component of DHIC which may influence the clinical presentation of incontinence and impede its management. Isolated DU was a rare primary cause of incontinence, but a common secondary diagnosis. Total DU prevalence in this population was 59%.

Pathophysiology

While precise DU etiology remains elusive, likely causes may include, but certainly are not be limited to, factors such as BPH, diabetes mellitus, and neurological problems involving the lumbosacral nerve roots. Advanced age may represent yet another major risk factor for retention. Among men with BPH followed in the Olmsted county study, the 5 year risk for acute retention rose from 1.6% in the 40-49 to 10% in the 70-79 age group. In population-based studies, peak flow rates decline with age independently of prostate volume and symptom severity. Finally, as for female nursing home residents who are incontinent, DU and DHIC are also very common among incontinent male nursing home residents who demonstrate no evidence of BOO.

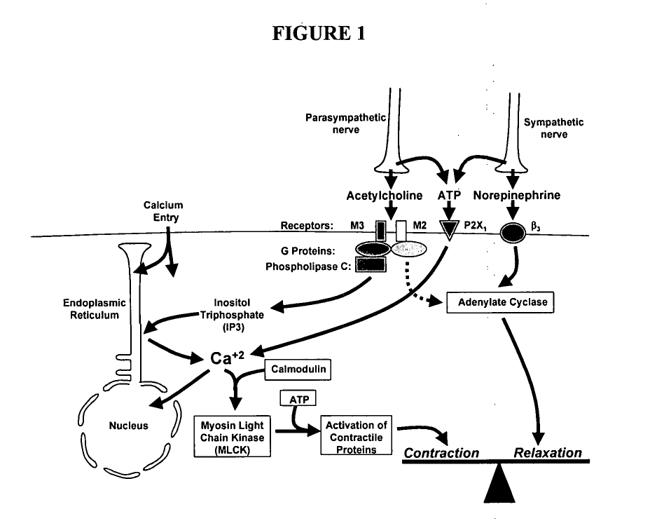

Bladder structure and plasticity. The human bladder is primarily composed of smooth muscle cells arranged into fascicles or bundles separated by an extracellular matrix containing collagen and elastin. While the bladder bears similarities to other contractile tissues containing smooth muscle, important differences also exist. Beyond barrier effects, uroepithelial cells also play important sensory roles, influence the survival and function of other bladder elements and may contribute to the pathogenesis of specific disorders. In most tissues, including the bladder trigone and urethra, smooth muscle cells within individual muscle bundles are well-organized. In contrast, bundles of detrusor smooth muscle cells generally run in all directions. Neurological bladder input is derived from sympathetic, parasympathetic and somatic systems (FIG. 1). Parasympathetic (S2-S4) sacral nerve roots mediate detrusor muscle contraction and relaxation of urethral muscle. Sympathetic lumbar nerves inhibit detrusor muscle contractions, while increasing internal sphincter tone, resulting in elevated bladder outlet resistance. Somatic input via pudendal nerves activates the external urethral sphincter.

The adult and aged bladder exhibits unexpected plasticity. For example, caveolae are vesicles involved in cellular signaling which are abundant within the sarcolemmal (submembrane) regions of adult bladder muscle cells. Their numbers increase with bladder maturation, but decrease during aging, estrogen depletion and injury. Even more striking plasticity develops in bladders distended in partial BOO. During a compensatory phase, normal bladder emptying is maintained with an increased bladder muscle mass, mostly due to hypertrophy of individual muscle cells. During a subsequent decompensation phase, the bladder undergoes muscle loss, collagen infiltration and axonal degeneration.

Normal bladder emptying. Micturition can be viewed as a simplistic process alternating between urine storage and expulsion. Layered upon this are complex central and peripheral neural pathways which enhance, inhibit and coordinate bladder performance. Detrusor muscle is relaxed during filling with sympathetic input allowing accommodation of increasing volumes of urine without notable change in wall tension. Sphincters are closed and the urethra is collapsed. This permits urinary continence and the ability to initiate micturition when physiologically and socially acceptable. Once voiding is initiated through cessation of sympathetic and somatic input plus concomitantly increased parasympathetic activity, sphincters relax, the bladder neck funnels and the detrusor generates pressure for overcoming resistance generated by the urethra. Signaling pathways regulating detrusor performance are illustrated in FIG. 1.

Physiology of declining detrusor contractility with aging. Normal aging, common medications and diverse disease processes may interfere with the signaling pathways highlighted in FIG. 1, hindering normal detrusor contractions. The “dense band” pattern with caveolar depletion has been described as the ultrastructural pattern associated with normative bladder aging (Table 2), lack of estrogen and injury. Muscarinic and purinergic receptors are among the signaling molecules known to cluster in caveolae. Studies in mice rendered null for the caveolin-1 gene demonstrate a disruption in the ability of cholinergic agonists to mediate bladder muscle contraction. It remains to be seen whether caveolar depletion represents a potentially reversible contributor to the declines in detrusor contractility observed with aging and the menopause.

TABLE 2
Ultrastructural features of bladder aging and of detrusor underactivity
in human and animal studies

| | Normative | Detrusor |
|---|---|---|
| | Aging | Underactivity |
| EM Pattern | “Dense Band” | “Degeneration” |
| Muscle Compartment | “De-differentiation” | Muscle Loss and |
| | (loss of caveolae) | Degeneration |
| Extra-Cellular Matrix | Widened ECM with mild | Widened ECM with |
| Compartment | collagen deposits | extensive collagen |
| (ECM) | | deposition |
| Axonal | Intact | Axonal |
| Fibers | | Degeneration |

Pathophysiology of Detrusor Underactivity

DU represents a greater decline in detrusor performance than has been described as part of normative aging. In attempts to define its etiology, some researchers have performed muscle strip contractility studies, while others have examined ultrastructural detrusor changes. Elbadawi et al. reported consistent degenerative findings in bladder muscle biopsies from older subjects with DU. In patients with pure DU, defined by Schäfer's criteria or as an unstrained PVR of >50 ml, electron microscopy revealed widespread muscle cell and axonal degeneration and widening of the interstitial spaces with collagen deposition. In contrast to a controversy regarding the nature of ultrastructural changes associated with detrusor over activity, the relationship of these types of degenerative changes to DU has been replicated by several groups in the setting of “idiopathic” DU, bladder outlet obstruction and also diabetic cystopathy. It has been reported that individuals with greater numbers of disruptive cells were more likely to demonstrate isolated DU or DU in the setting of BOO. In individuals with DU and BOO, and in those with evidence of both DU and detrusor over activity (DHIC), these degenerative changes are also accompanied by the presence of the myohypertrophy or the dysjunction ultrastructural patterns, respectively.

The multifactorial nature of detrusor underactivity. Abnormal bladder distension induces smooth muscle damage, muscle loss and fibrosis in animal models and could similarly promote DU in humans. Similarly, a lack of estrogen could contribute to the progression of DU. Estrogens promote the survival of smooth muscle cells and neurons. Ultrastructural changes described with DU and other changes associated with “normative” human aging have also been noted in bladders from ovariectomized rats (Table 2). Thus, a lack of estrogen could contribute to DU at several different levels including a decline in levels of contractile muscle proteins, loss of caveolae with a resulting decrease in intracellular calcium entry, as well as axonal and muscle degeneration. In spite of these basic studies, there is no compelling human evidence to implicate the menopause or estrogen declines in DU. None of the urodynamic studies involving elderly subjects included hormonal measurements and randomized trials of estrogen replacement did not include DU or PVR assessments. Endogenous estrogens remain detectable in older women and men. Their levels vary greatly between individuals, continuing to exert biological effects into late life. To date, only one published study has examined the relationship between estrogen status and PVR in older women. In a retrospective case-controlled study of 204 ambulatory women with a mean age of 79 years (range 62 to 94), authors concluded that the mean PVR was higher in women not receiving estrogen when compared to estrogen users (66.7 ml vs. 39.3 ml; p=0.002). Although this relationship was statistically significant and appeared to be independent of UTI, the physiologic and clinical significance of this observation remains unclear. A large trial showed that estrogen replacement does not lower, but rather raises the risk and severity of incontinence in postmenopausal women.

It has also been recently reported that while in younger individuals detrusor over activity is associated with evidence of enhanced detrusor contractility, this relationship is not observed in older subjects. It has been proposed that if estrogen replacement not only prevented declines in contractility associated with voluntary detrusor contractions, but also strengthened contractions resulting from detrusor over activity, then women on estrogen could become more vulnerable to leakage during urgency. Finally, aging itself is a significant risk factor for DU and DHIC. Modest aging-related declines in detrusor contractility may contribute to such vulnerability, while detrusor responses to physiological challenges may change with age. For example, older men with BPH are more likely to develop urinary retention, while aged rat and rabbit detrusors demonstrate a diminished capacity to maintain adequate pressures in response to BOO. It has been proposed that decreased mitochondrial metabolism and/or anoxic mechanisms could contribute to this deficit.

Management Issues

Catheter use. Catheters play a role in managing DU in older adults, yet clinical indications are circumscribed, with intermittent catheterization nearly always preferable to indwelling catheters. PVR measurements allow the clinician to diagnose acute or chronic urinary retention and may even offer insights into the presence of DU. Increasing numbers of clinical facilities and practices are purchasing ultrasound machines since ultrasound PVR assessments are non-invasive and should, in most cases represent the first step in diagnosing urinary retention. However, catheterizations for this purpose are safe, while also providing an optimal sample for culture and allowing for bladder decompression. Acute urinary retention is a common urological emergency with a measurable impact on patients' quality of life and substantial economic costs. Its immediate management, particularly when the bladder volume exceeds 1 liter, nearly always involves catheter placement. Many clinicians favor decompressing the bladder for a week or more in order to improve the likelihood of a successful voiding trial. Animal studies indicate that retention results in detrusor damage, collagen infiltration and decreased contractility. Many of these changes improve following relief of obstruction. It has been proposed that bladder decompression may facilitate healing and promote the restoration of normal detrusor contractility. Concerns about post-obstructive diuresis, hematuria and hypotension have led to suggestions that gradual decompression is preferable, yet these complications are rare and indwelling catheter clamping should be avoided. Individuals discharged with a short-term indwelling catheter following acute retention may maintain their previous activities of daily living.

Long-term use of indwelling catheters can be associated with a high risk of complications including urinary tract infections, metal erosions in men and vaginal fistulas in women. Geriatricians need to be vigilant since 5-10% of nursing home residents and about 4% of home care patients use indwelling catheters. Surveyor guidance for catheter use recently issued by Centers for Medicare and Medicaid Services highlights the need to use indwelling catheters judiciously, yet occasionally longer-term catheter use may be unavoidable. It has been proposed, but remains unproven, that PVRs greater than 50% of bladder capacity are unacceptable. In patients with spinal cord injury, intermittent catheterization is optimal. However, best-practice management of elevated PVRs in older adults without relevant neurological disease is less clear, as is the treatment of urinary tract infections in the setting of elevated PVR. In the presence of urinary retention, antibiotics alone may not be effective, given the bladder's inability to empty completely. Moreover, antibiotic use in the presence of an indwelling catheter leads to resistant nosocomial infection. The use of antibiotics combined with intermittent catheterization represents a sensible alternative. Interestingly, CMS guidance to surveyors recommends sterile technique when performing intermittent catheterization in the long term care setting, although sterile technique is not required when performing intermittent catheterization in the community.

Bethanechol. Bethanechol chloride (Urecholine®), a cholinergic agonist, is widely prescribed for urinary retention. When administered subcutaneously at higher doses it may favorably alter bladder performance. However, in randomized placebo controlled trials, it does not improve clinically-relevant outcomes. Normally, axonal degeneration is followed by denervation supersensitivity and increased responsiveness to cholinergic agonists, yet DU is also associated with muscle cell degeneration which most likely contributes to bethanechol's ineffectiveness. While it has been proposed that bethanechol may improve voiding in individuals with intact detrusor muscle such as those in whom contractility had been impaired by pharmacologic blockade involving potent anticholinergics, based on currently available evidence this medication cannot be recommended for the management of DU.

Many medications can decrease detrusor contractility, promoting the onset of DU and retention by pharmacologically decreasing detrusor contractility. However, the data linking specific medications to DU are sparse and not always clinically relevant. In one population study, age-adjusted odds ratio (OR; 95% CI) for incident acute urinary retention was elevated for baseline use of several medications. Antiarrhythmics (3.3; 1.1-9.3), non-diuretic hypertensives (2.8; 1.5-5.3), calcium channel antagonists (2.2; 1.2-3.9) and beta-blockers (1.9; 1.1-3.3) were shown to increase the odds ratio for acute retention in older men, yet effects of these medications on retention could be mediated via increased BOO and/or decreased detrusor contractility. In earlier studies, one potent antimuscarinic agent (atropine) failed to influence bladder function, while another (propantheline) resulted in acute urinary retention in 5/6 men with BPH. Most antispasmodics used for the treatment of urge incontinence have anticholinergic properties, raising concerns about their use in older individuals with DU.

Other agents represent rarer contributors to urinary retention. For example, botulinum toxin precipitates both paralysis and urinary retention by inhibiting acetylcholine release via presynaptic inhibition. One report has associated recent use of non-steroidal anti-inflammatory drugs with onset of acute retention. Other compounds ranging from erythromycin to St John's Wort (Hypericum perforatum) inhibit detrusor contractility in vitro, yet the clinical relevance of muscle strip studies, particularly when high drug concentrations are studied, remains unclear. Case reports of urinary retention induced by potent psychoactive drugs (fluoxetine-risperidone) and sympathomimetic recreational drugs (ecstasy, methamphetamine) also exist. In spite of a limited clinical literature, all medications with a demonstrated or potential ability to decrease detrusor contractility, including over-the-counter cold medicines containing anticholinergic antihistamines with sympathomimetics, should be used with caution and must be discontinued in acute or worsening urinary retention.

DU and Detrusor Over activity (DHIC). The optimal treatment of individuals who have evidence of both DU and detrusor over activity (DHIC) remains unknown. It is unclear whether pharmacologic agents currently used for treating symptoms associated with an overactive bladder are equally effective and safe when DU is also present. Frail elderly with DHIC may be more susceptible to develop anticholinergic side effects including worsening cognition, constipation or xerostomia. The presence of DU in DHIC may also increase the risk of antispasmodic-induced chronic or even acute retention. A 8 week double-blind randomized placebo-controlled trial examined the safety and effectiveness of oxybutynin in 110 community-dwelling older adults whose incontinence was primarily due to detrusor over activity, who were cognitively intact and also demonstrated evidence of DHIC. Immediate-release oxybutynin started at 2.5 mg and was titrated upward based on efficacy and side effects. On intention-to-treat analysis, daily incontinence frequency decreased by 68% in 64 subjects randomized to oxybutynin versus 40% randomized to placebo (p=0.004). No incontinence episodes occurred on the end-study bladder diary in 34/64 subjects randomized to oxybutynin versus 7/46 randomized to placebo (p<0.0005). However, on oxybutynin, 25/35 (71%) of evaluable subjects with normal contractility had no end-study leakage, compared with 9/19 (47%) with impaired contractility (p=0.08). Overall, the use of oxybutynin was surprisingly safe in this population with no evidence of acute retention and with only one subject requiring dose adjustment during titration (for PVR>400 ml). Nevertheless, the long-term consequences, effectiveness and safety of antispasmodic use in those with more severe DU, especially those who are frail, cognitively impaired and residing in long-term care institutions, remains unknown. In rare individuals, urge symptoms may only be relieved with antispasmodic doses which induce complete retention, requiring intermittent catheterization. It remains to be seen whether M3-selective antispasmodics or behavioral approaches involving bladder training and pelvic floor muscle training assisted with biofeedback could offer advantages in some elderly subjects with DU associated with urge or stress incontinence.

Hip fractures and other post-operative settings. Urinary retention may develop after any surgery, yet hip fracture repair and total joint (hip, knee) arthroplasty carry a particularly high risk, while the risk associated with hernia or anal surgery is intermediate. Staff must be vigilant since as many as two-thirds of post-operative subjects with a PVR greater than 600 ml reported neither symptoms of discomfort nor a strong urge to void. As in other subjects with urinary retention, efforts must be made to reverse or remove all potential contributors. There is no evidence that particular types of anesthesia offer specific benefits or disadvantages.

Hip fractures represent a particularly important risk factor for urinary retention. Skelly et al., found that 67/69 (97%) of older individuals with hip fracture had catheterized PVRs>150 ml within 8 hours of admission. In a prospective study of 309 elderly women with a new proximal femoral fracture, ultrasound evidence of elevated PVRs (>300 ml) was present in 79% on admission to hospital, in 37% pre-operatively, in 56% during the first 24 hours after surgery and in 22% during the recovery phase. Advanced age is a risk for elevated PVRs pre-operatively, for a decreased ability to lower PVRs during the recovery phase and for requiring straight catheterization for retention. The high prevalence of retention in this population has already led to the routine use of indwelling urinary catheters during the peri-operative period.

Careful catheter use may play a role in helping patients regain voiding following orthopedic surgery. In a randomized study of 100 patients undergoing total hip or knee replacement, Michelson et al. demonstrated that the routine short-term use of indwelling catheters reduced the risk of urinary retention and bladder overdistension without increasing infection risk when compared to intermittent catheterization as needed. While many patients were 60 years and older, they were selected to undergo an elective arthroplasty. Moreover, a third were younger, operated for avascular hip necrosis. In a study of 67 elderly women and men who had suffered a hip fracture, Skelly et al. concluded that satisfactory voiding was achieved more rapidly with intermittent catheterization as compared to the placement of an indwelling catheter. In this study intermittent catheterization was performed on a regular schedule, with the frequency increased in order to avoid overdistension and the protocol followed until PVR was less than 150 ml on at least two separate occasions. This is in contrast to most other studies including that by Michelson et al. in which catheterization was performed only if the patients were unable to void, with catheterized volumes ranging from 800 to 1000 ml. A rigorous regular catheterization protocol is quite labor-intensive and costly. Moreover, to date, no published trial has addressed optimum care in cognitively impaired and/or institutionalized individuals.

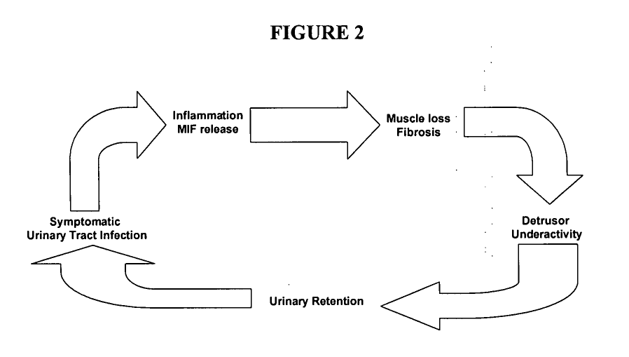

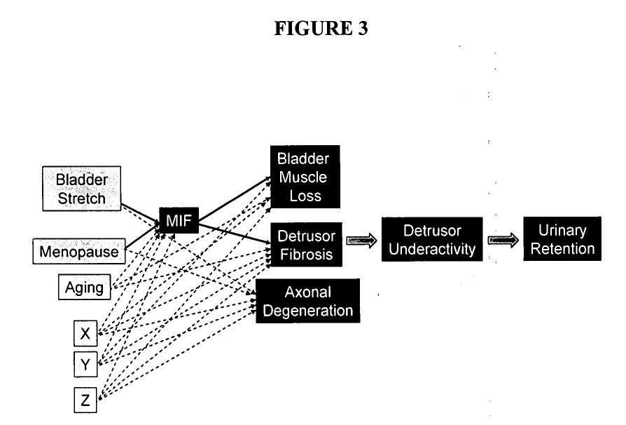

The pathophysiology of urinary retention in these individuals remains to be defined. Pelvic injury and pain from the fracture and from surgery could hinder bladder emptying. These individuals also become immobile and are at high risk of constipation, while suffering from multiple morbidities and taking many medications which adversely impact detrusor function. Basic research suggests a potential pathophysiologic framework in which local inflammatory events, lack of estrogen and urinary retention may all interact in promoting DU. It remains to be seen whether pro-inflammatory mediators released by local trauma or urinary tract infections associated with retention, low estrogen levels known to be linked to a higher hip fracture risk and/or the mechanical effect of urinary retention itself may contribute to the pathogenesis of DU in selected clinical contexts (FIGS. 2, 3; Table 1).

Relationship to Mobility Disorders, Frailty and Delirium. DU and DHIC are geriatric conditions which are especially common in the frail elderly. Hospitalized and institutionalized older adults with urinary retention are generally less mobile and more dependent. Declines in physical performance predict future disability and reduced longevity, yet no conclusions can be drawn regarding links between urinary retention or DU and frailty as defined by Fried et al. Among elderly at risk for DU and DHIC, cognitive deficits are also common. Anticholinergic agents may worsen both detrusor contractility and cognitive function in vulnerable elderly. “Cystocerebral syndrome” may represent a form of delirium provoked by acute urinary retention and resolved with bladder decompression. Thus, as in the case of other geriatric syndromes, some risk factors for DU and for cognitive deficits (dementia, delirium) might also be shared.

MIF, a pro-inflammatory urothelium-derived cytokine, has been implicated in the development of bladder muscle loss and fibrosis. Moreover, infectious and inflammatory stimuli have been shown to enhance the release of MIF, which is in turn inhibited by very low concentrations of estrogen. The present invention, therefore, relates to the discovery that MIF and related molecules participate in the development of a vicious cycle with chronic infection and inflammation promoting greater retention which, in turn, makes urinary tract infection more likely and more difficult to eradicate. Post-menopausal estrogen depletion could accelerate these events, while the beneficial effects of intravaginal estrogen on recurrent infections in post-menopausal women may be mediated, at least in part, via estrogen's ability to downregulate MIF.

Biopsy studies have established a strong relationship between urodynamic evidence of DU and structural changes including detrusor fibrosis, muscle degeneration and axonal degeneration. Moreover, the development of these changes in the setting of BOO correlates with symptom severity and may herald the development of DU. Animal studies have provided a wealth of information regarding cellular events which are associated with detrusor muscle hypertrophy that occurs as part of the compensatory response to BOO. Nevertheless, a complicating factor in being able to directly implicate specific molecules in these compensatory events has been the fact that mouse deletions of some of the most promising candidate genes (e.g. TGF-β, TGF-β-R, cyr61, CTGF and FGF-10) are lethal. An alternative approach is one which seeks to define mechanisms involved in the development of detrusor decompensation.

Macrophage migration inhibitory factor (MIF) is a potent pro-inflammatory cytokine which has been shown to be a key mediator of bladder inflammation. This protein is abundantly expressed in urothelial cells (i.e., the layer of mucosal epithelial cells lining the bladder) and is known to be released by varied inflammatory stimuli. MIF was discovered as a potent inhibitor of random macrophage migration which enhances macrophage retention and tissue inflammation. While MIF enhances fibroblast survival, it also promotes cardiomyocyte death in vivo. A detailed description of MIF can be found in U.S. Pat. No. 6,645,493 to Bucala et al. which is incorporated herein by reference in its entirety for all purposes.

Diagnostic Oligonucleotides of the Invention

As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

In certain aspects, the invention relates to diagnostic oligonucleotides and diagnostic oligonucleotide set(s), for which a correlation exists between the health status of an individual, and the individual's expression of RNA or protein products corresponding to the nucleotide sequence. In some instances, only one oligonucleotide is necessary for such detection. Members of a diagnostic oligonucleotide set may be identified by any means capable of detecting expression or a polymorphism of RNA or protein products, including but not limited to differential expression screening, PCR, RT-PCR, SAGE analysis, high-throughput sequencing, microarrays, liquid or other arrays, protein-based methods (e.g., western blotting, proteomics, mass-spectrometry, and other methods described herein), and data mining methods, as further described herein.

In the context of the invention, nucleic acids and/or proteins are manipulated according to well known molecular biology techniques. Detailed protocols for numerous such procedures are described in, e.g., in Ausubel et al. Current Protocols in Molecular Biology (supplemented through 2000) John Wiley & Sons, New York (“Ausubel”); Sambrook et al. Molecular Cloning—A Laboratory Manual (2nd Ed.), Vol. 1-3, Cold Spring Harbor Laboratory, Cold Spring Harbor, N.Y., 1989 (“Sambrook”), and Berger and Kimmel Guide to Molecular Cloning Techniques, Methods in Enzymology volume 152 Academic Press, Inc., San Diego, Calif. (“Berger”).

Genotyping

In addition to, or in conjunction with the correlation of expression profiles and clinical data, it is often desirable to correlate expression patterns with the subject's genotype at one or more genetic loci or to correlate both expression profiles and genetic loci data with clinical data. The selected loci can be, for example, chromosomal loci corresponding to one or more member of the candidate library, polymorphic alleles for marker loci, or alternative disease related loci (not contributing to the candidate library) known to be, or putatively associated with, a disease (or disease criterion). Indeed, it will be appreciated, that where a (polymorphic) allele at a locus is linked to a disease (or to a predisposition to a disease), the presence of the allele can itself be a disease criterion.

Numerous well known methods exist for evaluating the genotype of an individual, including southern analysis, restriction fragment length polymorphism (RFLP) analysis, polymerase chain reaction (PCR), amplification length polymorphism (AFLP) analysis, single stranded conformation polymorphism (SSCP) analysis, single nucleotide polymorphism (SNP) analysis (e.g., via PCR, Taqman or molecular beacons), among many other useful methods. Many such procedures are readily adaptable to high throughput and/or automated (or semi-automated) sample preparation and analysis methods. Most, can be performed on nucleic acid samples recovered via simple procedures from the same sample as yielded the material for expression profiling. Exemplary techniques are described in, e.g., Sambrook, and Ausubel, supra.

The invention also features nucleic acid molecules, for example enzymatic nucleic acid molecules, antisense nucleic acid molecules, decoys, double stranded RNA, triplex oligonucleotides, and/or aptamers, and methods to modulate gene expression of, for example, genes encoding a MIF protein, a MIF binding protein or a MIF receptor protein. In particular, the instant invention features nucleic-acid based molecules and methods to modulate the expression of a MIF protein or MIF receptor protein.

The invention features one or more enzymatic nucleic acid-based molecules and methods that independently or in combination modulate the expression of gene(s) encoding a MIF protein (Genbank Accession No. CAG30406), a MIF binding protein, and/or a MIF receptor protein, for example, CD44 and/or CD74 (Genbank Accession Nos. NP_{—}000601, and NP_{—}001020330, respectively).

The description below of the various aspects and embodiments is provided with reference to the exemplary MIF and MIF receptor genes. However, the various aspects and embodiments are also directed to genes which encode homologs, orthologs, and paralogs of other MIF proteins, MIF binding proteins, and MIF receptor genes and include all isoforms, splice variants, and polymorphisms. Those additional genes can be analyzed for target sites using the methods described for MIF proteins, MIF binding proteins, and MIF receptor genes. Thus, the inhibition and the effects of such inhibition of the other genes can be performed as described herein.

By “down-regulate” it is meant that the expression of the gene, or level of RNAs or equivalent RNAs encoding one or more proteins, or activity of one or more proteins, such as MIF and MIF receptor genes, is reduced below that observed in the absence of the nucleic acid molecules of the invention. In one embodiment, inhibition or down-regulation with enzymatic nucleic acid molecule preferably is below that level observed in the presence of an enzymatically inactive or attenuated molecule that is able to bind to the same site on the target RNA, but is unable to cleave that RNA. In another embodiment, inhibition or down-regulation with antisense oligonucleotides is preferably below that level observed in the presence of, for example, an oligonucleotide with scrambled sequence or with mismatches. In another embodiment, inhibition or down-regulation of MIF proteins, MIF binding proteins, and MIF receptor genes with the nucleic acid molecule of the instant invention is greater in the presence of the nucleic acid molecule than in its absence.

By “up-regulate” is meant that the expression of the gene, or level of RNAs or equivalent RNAs encoding one or more protein subunits, or activity of one or more protein subunits, such as MIF proteins, MIF binding proteins, and MIF receptor genes, is greater than that observed in the absence of the nucleic acid molecules of the invention. For example, the expression of a gene, such as MIF proteins, MIF binding proteins, and MIF receptor genes, can be increased in order to treat, prevent, ameliorate, or modulate a pathological condition caused or exacerbated by an absence or low level of gene expression. In one embodiment the invention relates to a method for treating or preventing bladder over activity by up-regulating the expression, release, and/or activity of a MIF proteins, MIF binding proteins, and MIF receptor genes.

By “modulate” is meant that the expression of the gene, or level of RNAs or equivalent RNAs encoding one or more proteins, or activity of one or more proteins is up-regulated or down-regulated, such that the expression, level, or activity is greater than or less than that observed in the absence of the nucleic acid molecules of the invention.

By “gene” it is meant a nucleic acid that encodes RNA, for example, nucleic acid sequences including but not limited to a segment encoding a polypeptide.

“Complementarity” refers to the ability of a nucleic acid to form hydrogen bond(s) with another RNA sequence by either traditional Watson-Crick or other non-traditional types.

By “RNA” is meant a molecule comprising at least one ribonucleotide residue. By “ribonucleotide” or “2′-OH” is meant a nucleotide with a hydroxyl group at the 2′ position of a D-ribo-furanose moiety.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetyltidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra).

By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “enzymatic nucleic acid molecule” it is meant a nucleic acid molecule which has complementarity in a substrate binding region to a specified gene target, and also has or mediates an enzymatic activity which is active to specifically cleave target RNA. That is, the enzymatic nucleic acid molecule is able to intermolecularly cleave RNA, alone or as a component of an enzymatic complex, and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner and Uhlenbeck, 1995, Nucleic Acids Research, 23, 2092 2096; Hammann et al., 1999, Antisense and Nucleic Acid Drug Dev., 9, 25 31). The nucleic acids can be modified at the base, sugar, and/or phosphate groups. The term “enzymatic nucleic acid” is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, siRNA, micro RNA, short hairpin RNA, endoribonuclease, RNA-induced silencing complexes, endonuclease, minizyme, leadzyme, oligozyme or DNA enzyme. All of these terminologies describe nucleic acid molecules with enzymatic activity.

The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

Several varieties of enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

By “nucleic acid molecule” as used herein is meant a molecule having nucleotides. The nucleic acid can be single, double, or multiple stranded and can comprise modified or unmodified nucleotides or non-nucleotides or various mixtures and combinations thereof.

By “equivalent” or “related” RNA to MIF proteins, MIF binding proteins, and MIF receptor genes is meant to include those naturally occurring—RNA molecules having homology (partial or complete) to MIF proteins, MIF binding proteins, and MIF receptor genes encoding for proteins with similar function as MIF proteins, MIF binding proteins, and MIF receptor proteins in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like. By “homology” is meant the nucleotide sequence of two or more nucleic acid molecules is partially or completely identical. In certain embodiments the homolgous nucleic acid has 30%, 40%, 50%, 60%, 70%, 80%, 90%, or 95% homology to MIF, MIF binding protein, and/or MIF receptor gene.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop or hairpin, and/or an antisense molecule can bind such that the antisense molecule forms a loop or hairpin. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol, 40, 1-49, which are incorporated herein by reference in their entirety. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

Long double-stranded RNAs (dsRNAs; typically >200 nt) can be used to silence the expression of target genes in a variety of organisms and cell types (e.g., worms, fruit flies, and plants). Upon introduction, the long dsRNAs enter a cellular pathway that is commonly referred to as the RNA interference (RNAi) pathway. First, the dsRNAs get processed into 20-25 nucleotide (nt) small interfering RNAs (siRNAs) by an RNase III-like enzyme called Dicer (initiation step). Then, the siRNAs assemble into endoribonuclease-containing complexes known as RNA-induced silencing complexes (RISCs), unwinding in the process. The siRNA strands subsequently guide the RISCs to complementary RNA molecules, where they cleave and destroy the cognate RNA (effecter step). Cleavage of cognate RNA takes place near the middle of the region bound by the siRNA strand. In mammalian cells, introduction of long dsRNA (>30 nt) initiates a potent antiviral response, exemplified by nonspecific inhibition of protein synthesis and RNA degradation. The mammalian antiviral response can be bypassed, however, by the introduction or expression of siRNAs.

Injection and transfection of dsRNA into cells and organisms has been the main method of delivery of siRNA. And while the silencing effect lasts for several days and does appear to be transferred to daughter cells, it does eventually diminish. Recently, however, a number of groups have developed expression vectors to continually express siRNAs in transiently and stably transfected mammalian cells. (See, e.g., Brummelkamp T R, Bernards R, and Agami R. (2002). A system for stable expression of short interfering RNAs in mammalian cells. Science 296:550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052, which are herein incorporated by reference in their entirety).

Some vectors have been engineered to express small hairpin RNAs (shRNAs), which get processed in vivo into siRNAs-like molecules capable of carrying out gene-specific silencing. The vectors contain the shRNA sequence between a polymerase III (pol III) promoter (e.g., U6 or H1 promoters) and a 4-5 thymidine transcription termination site. The transcript is terminated at position 2 of the termination site (pol III transcripts naturally lack poly(A) tails) and then folds into a stem-loop structure with 3′ UU-overhangs. The ends of the shRNAs are processed in vivo, converting the shRNAs into ˜21 nt siRNA-like molecules, which in turn initiate RNAi. This latter finding correlates with recent experiments in C. elegans, Drosophila, plants and Trypanosomes, where RNAi has been induced by an RNA molecule that folds into a stem-loop structure. The use of siRNA vectors and expression systems is known and are commercially available from Ambion, Inc.® (Austin, Tex.), Lentigen, Inc. (Baltimore, Md.), Panomics (Fremont, Calif.), and Sigma-Aldrich (ST. Louis, Mo.).

In another aspect of the invention, enzymatic nucleic acid molecules or antisense molecules that interact with target RNA molecules, and down-regulate MIF, MIF binding proteins, and/or a MIF receptor gene activity are expressed from transcription units inserted into DNA or RNA vectors. The recombinant vectors are preferably DNA plasmids or viral vectors. Enzymatic nucleic acid molecule or antisense expressing viral vectors can be constructed based on, but not limited to, lenti virus, cytomegalovirus, adeno-associated virus, retrovirus, adenovirus, or alphavirus. Preferably, the recombinant vectors capable of expressing the enzymatic nucleic acid molecules or antisense are delivered, and persist in target cells. Alternatively, viral vectors can be used that provide for expression of enzymatic nucleic acid molecules or antisense. Such vectors can be repeatedly administered as necessary. Once expressed, the enzymatic nucleic acid molecules or antisense bind to the target RNA and down-regulate its function or expression. Delivery of enzymatic nucleic acid molecule or antisense expressing vectors can be systemic, such as by intravenous or intramuscular administration, by administration to target cells explanted from the patient or subject followed by reintroduction into the patient or subject, or by any other means that would allow for introduction into the desired target cell. Antisense DNA can be expressed via the use of a single stranded DNA intracellular expression vector.

By “vectors” is meant any nucleic acid-based technique used to deliver a desired nucleic acid, for example, bacterial plasmid, viral nucleic acid, HAC, BAC, and the like.

The nucleic acid molecules of the instant invention, individually, or in combination or in conjunction with other drugs, can be used to treat diseases or conditions discussed above. For example, the subject can be treated, or other appropriate cells can be treated, as is evident to those skilled in the art, individually or in combination with one or more drugs under conditions suitable for the treatment.

The use of specially designed vector constructs for inducing RNA interference has numerous advantages over oligonucleotide-based techniques. The most significant advantages are stability, and induced transcription via inducible promoters. Promoter regions in the vector ensure that shRNA transcripts are constantly expressed, maintaining cellular levels at all times. Thus, the duration of the effect is not as transient as with injected RNA, which usually lasts no longer than a few days. And by using expression constructs instead of oligo injection, it is possible to perform multi-generational studies of gene knockdown because the vector can become a permanent fixture in the cell line.

By “triplex forming oligonucleotides” or “triplex oligonucleotide” is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504; Fox, 2000, Curr. Med. Chem., 7, 17-37; Praseuth et. al., 2000, Biochim. Biophys. Acta, 1489, 181-206).

By “double stranded RNA” or “dsRNA” is meant a double stranded RNA that matches a predetermined gene sequence that is capable of activating cellular enzymes that degrade the corresponding messenger RNA transcripts of the gene. These dsRNAs are referred to as short intervening RNA (siRNA) and can be used to inhibit gene expression (see for example Elbashir et al., 2001, Nature, 411, 494-498; and Bass, 2001, Nature, 411, 428-429). The term “double stranded RNA” or “dsRNA” as used herein refers to a double stranded RNA molecule capable of RNA interference “RNAi”, including short interfering RNA “siRNA” see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914.

The enzymatic nucleic acid molecule, antisense nucleic acid or other nucleic acid molecules of the invention that down regulate MIF, MIF binding proteins, and/or MIF receptor gene expression represent a therapeutic approach to treat a variety of bladder disorders and conditions, including but not limited to DU, DHIC, and any other condition which responds to the modulation of MIF and MIF receptor gene function. The use of inhibitory RNA molecules and techniques are known in the art and are described in detail in U.S. Pat. No. 7,022,828, the teachings of which are incorporated herein by reference in their entirety for all purposes.

In one embodiment of the present invention, a nucleic acid molecule of the instant invention can be between about 10 and 100 nucleotides in length. For example, enzymatic nucleic acid molecules of the invention are preferably between about 15 and 50 nucleotides in length, more preferably between about 25 and 40 nucleotides in length (for example see Jarvis et al., 1996, J. Biol. Chem., 271, 29107 29112). Exemplary antisense molecules of the invention are preferably between about 15 and 75 nucleotides in length, more preferably between about 20 and 35 nucleotides in length (see for example Woolf et al., 1992, PNAS., 89, 7305 7309; Milner et al., 1997, Nature Biotechnology, 15, 537 541). Exemplary triplex forming oligonucleotide molecules of the invention are preferably between about 10 and 40 nucleotides in length, more preferably between about 12 and 25 nucleotides in length (see for example Maher et al, 1990, Biochemistry, 29, 8820 8826; Strobel and Dervan, 1990, Science, 249, 73 75). Those skilled in the art will recognize that all that is required is that the nucleic acid molecule be of sufficient length and suitable conformation for the nucleic acid molecule to interact with its target and/or catalyze a reaction contemplated herein. The length of the nucleic acid molecules of the instant invention are not limiting within the general limits stated. Preferably, a nucleic acid molecule that modulates, for example, down-regulates MIF, MIF binding protein, and/or a MIF receptor gene expression comprises between 12 and 100 bases complementary to a RNA molecule of a MIF gene, a MIF binding protein gene, and/or a MIF receptor gene.

The invention provides a method for producing a class of nucleic acid-based gene modulating agents which exhibit a high degree of specificity for the RNA of a desired target. For example, the enzymatic nucleic acid molecule is preferably targeted to a highly conserved sequence region of target RNAs encoding a MIF, MIF binding protein, and/or a MIF receptor gene such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and/or RNA vectors that are delivered to specific cells.

As used in herein “cell” is used in its usual biological sense, and does not refer to an entire multicellular organism. The cell can, for example, be in vivo, in vitro or ex vivo, e.g., in cell culture, or present in a multicellular organism, including, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

By “MIF,” “MIF binding protein,” and “MIF receptor” proteins is meant, a peptide or protein comprising a full length MIF, MIF binding protein or a MIF receptor protein, domain, fusion protein, chimera, or fragment thereof.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues in vitro, ex vivo, or in vivo through injection or infusion pump, with or without their incorporation in biopolymers.

In another embodiment, the invention features an enzymatic nucleic acid molecule having one or more non-nucleotide moieties, and having enzymatic activity to cleave an RNA or DNA molecule.

In a further embodiment, the described nucleic acid molecules, such as antisense or ribozymes, can be used in combination with other known treatments to treat conditions or diseases discussed above. For example, the described molecules can be used in combination with one or more known therapeutic agents.

Antisense molecules can be modified or unmodified RNA, DNA, or mixed polymer oligonucleotides and primarily function by specifically binding to matching sequences resulting in inhibition of peptide synthesis (Wu-Pong, November 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA can result in nuclease degradation of the heteroduplex (Wu-Pong, supra; Crooke, supra). To date, the only backbone modified DNA chemistry which acts as substrates for RNase H are phosphorothioates, phosphorodithioates, and borontrifluoridates. Recently it has been reported that 2′-arabino and 2′-fluoro-arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., U.S. Ser. No. 60/101,174 which was filed on Sep. 21, 1998) all of these are incorporated by reference herein in their entirety.

Several varieties of enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83 87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakacane & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

The enzymatic nature of an enzymatic nucleic acid molecule can allow the concentration of enzymatic nucleic acid molecule necessary to affect a therapeutic treatment to be lower. This reflects the ability of the enzymatic nucleic acid molecule to act enzymatically. Thus, a single enzymatic nucleic acid molecule is able to cleave many molecules of target RNA. In addition, the enzymatic nucleic acid molecule is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to greatly attenuate the catalytic activity of a enzymatic nucleic acid molecule.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and achieve efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving enzymatic nucleic acid molecules can be used as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250).

Enzymatic nucleic acid molecules of the invention that are allosterically regulated (“allozymes”) can be used to modulate MIF, MIF binding proteins, and/or MIF receptor gene expression. These allosteric enzymatic nucleic acids or allozymes (see for example George et al, U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842) are designed to respond to a signaling agent, which in turn modulates the activity of the enzymatic nucleic acid molecule and modulates expression of MIF, MIF binding proteins, and/or MIF receptor gene. In response to interaction with a predetermined signaling agent, the allosteric enzymatic nucleic acid molecule's activity is activated or inhibited such that the expression of a particular target is selectively down-regulated. The target can comprise MIF, MIF binding proteins, and/or MIF receptor gene.

Oligonucleotides (eg; antisense, GeneBlocs) are synthesized using protocols known in the art as described in Caruthers et al., 1992, Methods in Enzymology 211, 3 19, Thompson et al., International PCT Publication No. WO 99/54459, Wincott et al., 1995, Nucleic Acids Res. 23, 2677 2684, Wincott et al., 1997, Methods Mol. Bio., 74, 59, Brennan et al, 1998, Biotechnol Bioeng., 61, 33 45, and Brennan, U.S. Pat. No. 6,001,311. All of these references are incorporated herein by reference. In a non-limiting example, small scale syntheses are conducted on a 394 Applied Biosystems, Inc. synthesizer. Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention can be modified extensively to enhance stability by modification with nuclease resistant groups, for example, 2′-amino, 2′-C-allyl, 2′-fluoro, 2′-O-methyl, 2′-H (for a review see Usman and Cedergren, 1992, TIBS 17, 34; Usman et al., 1994, Nucleic Acids Symp. Ser. 31, 163).

While chemical modification of oligonucleotide internucleotide linkages with phosphorothioate, phosphorothioate, and/or 5′-methylphosphonate linkages improves stability, too many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications that maintain or enhance activity are provided. Such nucleic acid is also generally more resistant to nucleases than unmodified nucleic acid. Nucleic acid molecules are preferably resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211, 3-19 (incorporated by reference herein) have expanded the ability to modify nucleic acid molecules by introducing nucleotide modifications to enhance their nuclease stability as described above. The use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules and/or other chemical or biological molecules). The treatment of subjects with nucleic acid molecules can also include combinations of different types of nucleic acid molecules.

In one embodiment, nucleic acid catalysts having chemical modifications that maintain or enhance enzymatic activity are provided. Such nucleic acids are also generally more resistant to nucleases than unmodified nucleic acid.

In one embodiment, the invention features modified enzymatic nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331 417, and Mesmaeker et al., 1994, Novel Backbone Replacements for Oligonucleotides, in Carbohydrate Modifications in Antisense Research, ACS, 24 39. These references are hereby incorporated by reference herein. Various modifications to nucleic acid (e.g., antisense and ribozyme) structure can be made to enhance the utility of these molecules. For example, such modifications can enhance shelf-life, half-life in vitro, bioavailability, stability, and ease of introduction of such oligonucleotides to the target site, including e.g., enhancing penetration of cellular membranes and conferring the ability to recognize and bind to targeted cells.

Administration of Nucleic Acid Molecules. Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by a incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example, through the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. NeuroVirol., 3, 387-400.

The molecules of the instant invention can be used as pharmaceutical agents. Pharmaceutical agents prevent, inhibit the occurrence, or treat (alleviate a symptom to some extent, preferably all of the symptoms) a disease state in a subject.

The negatively charged polynucleotides of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or subject, preferably a human. By “systemic administration” is meant in vivo systemic absorption or accumulation of drugs in the blood stream followed by distribution throughout the entire body. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

Administration routes which lead to systemic absorption include, without limitations: intravenous, subcutaneous, intraperitoneal, inhalation, oral, intrapulmonary and intramuscular. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of nucleic acid molecules include material described in Boado et al., 1998, J. Pharm. Sci., 87, 1308-1315; Tyler et al, 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 1000 mg/kg body weight/day of active ingredients is administered dependent upon potency of the negatively charged polymer.

The formulations can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

Nucleic acid molecules of the invention can also be administered in the form of suppositories, e.g., for rectal administration of the drug or via a catheter directly to the bladder itself. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

The amount of active ingredient that can be combined with the carrier materials to produce a single dosage form varies depending upon the host treated and the particular mode of administration. Dosage unit forms generally contain between from about 1 mg to about 1000 mg of an active ingredient.

It is understood that the specific dose level for any particular patient or subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The composition can also be administered to a subject in: combination with other therapeutic compounds to increase the overall therapeutic effect. The use of multiple compounds to treat an indication can increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591 5; Kashani-Sabet et al., 1992, Antisense Res. Dev., 2, 3 15; Dropulic et al., 1992, J. Virol., 66, 1432 41; Weerasinghe et al., 1991, J. Virol., 65, 5531 4; Ojwang et al., 1992, Proc. Natl. Acad. Sci. USA, 89, 10802 6; Chen et al., 1992, Nucleic Acids Res., 20, 4581 9; Sarver et al., 1990 Science, 247, 1222 1225; Thompson et al, 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector.

In one aspect the invention features an expression vector comprising a nucleic acid sequence encoding at least one of the nucleic acid molecules of the instant invention. The nucleic acid sequence encoding the nucleic acid molecule of the instant invention is operably linked in a manner which allows expression of that nucleic acid molecule.

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743 7; Gao and Huang 1993, Nucleic Acids Res., 21, 2867 72; Lieber et al., 1993, Methods Enzymol., 217, 47 66; Zhou et al., 1990, Mol. Cell. Biol., 10, 4529 37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3 15; Ojwang et al., 1992, Proc. Natl. Acad. Sci. USA, 89, 10802 6; Chen et al, 1992, Nucleic Acids Res., 20, 4581 9; Yu et al., 1993, Proc. Natl. Acad. Sci. USA, 90, 6340 4; L'Huillier et al., 1992, EMBO J., 11, 4411 8; Lisziewicz et al., 1993, Proc. Natl. Acad. Sci. U.S.A, 90, 8000 4; Thompson et al., 1995, Nucleic Acids Res., 23, 2259; Sullenger & Cech, 1993, Science, 262, 1566).

In another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

A further object of the present invention is to provide a kit comprising a suitable container, the therapeutic of the invention in a pharmaceutically acceptable form disposed therein, and instructions for its use.

In another embodiment, an isolated nucleic acid molecule of the invention comprises a nucleic acid molecule that is a complement of the nucleotide sequence of MIF, a MIF binding protein, and/or a MIF receptor. As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect.

As used herein, “fragments” are defined as sequences of at least 6 (contiguous) nucleic acids or at least 4 (contiguous) amino acids, a length sufficient to allow for specific hybridization in the case of nucleic acids or for specific recognition of an epitope in the case of amino acids, and are at most some portion less than a full length sequence.

The term “host cell” includes a cell that might be used to carry a heterologous nucleic acid, or expresses a peptide or protein encoded by a heterologous nucleic acid. A host cell can contain genes that are not found within the native (non-recombinant) form of the cell, genes found in the native form of the cell where the genes are modified and re-introduced into the cell by artificial means, or a nucleic acid endogenous to the cell that has been artificially modified without removing the nucleic acid from the cell. A host cell may be eukaryotic or prokaryotic. General growth conditions necessary for the culture of bacteria can be found in texts such as BERGEY'S MANUAL OF SYSTEMATIC BACTERIOLOGY, Vol. 1, N. R. Krieg, ed., Williams and Wilkins, Baltimore/London (1984). A “host cell” can also be one in which the endogenous genes or promoters or both have been modified to produce one or more of the polypeptide components of the complex of the invention.

“Derivatives” are compositions formed from the native compounds either directly, by modification, or by partial substitution.

“Analogs” are nucleic acid sequences or amino acid sequences that have a structure similar to, but not identical to, the native compound.

Derivatives or analogs of the nucleic acids or proteins of the invention include, but are not limited to, molecules comprising regions that are substantially homologous to the nucleic acids or proteins of the invention, in various embodiments, by at least about 30%, 40%, 50%, 60%, 70%, 80%, 90%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins of the invention under stringent, moderately stringent, or low stringent conditions. See e.g. Ausubel, et al., CURRENT PROTOCOLS 1N MOLECULAR BIOLOGY, John Wiley & Sons, New York, N.Y., 1993. Nucleic acid derivatives and modifications include those obtained by gene replacement, site-specific mutation, deletion, insertion, recombination, repair, shuffling, endonuclease digestion, PCR, subcloning, and related techniques.

“Homologs” can be naturally occurring, or created by artificial synthesis of one or more nucleic acids having related sequences, or by modification of one or more nucleic acid to produce related nucleic acids. Nucleic acids are homologous when they are derived, naturally or artificially, from a common ancestor sequence (e.g., orthologs or paralogs). If the homology between two nucleic acids is not expressly described, homology can be inferred by a nucleic acid comparison between two or more sequences. If the sequences demonstrate some degree of sequence similarity, for example, greater than about 30% at the primary amino acid structure level, it is concluded that they share a common ancestor. For purposes of the present invention, genes are homologous if the nucleic acid sequences are sufficiently similar to allow recombination and/or hybridization under low stringency conditions.

As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA.

Furthermore, one of ordinary skill will recognize that “conservative mutations” also include the substitution, deletion or addition of nucleic acids that alter, add or delete a single amino acid or a small number of amino acids in a coding sequence where the nucleic acid alterations result in the substitution of a chemically similar amino acid. Amino acids that may serve as conservative substitutions for each other include the following: Basic: Arginine (R), Lysine (K), Histidine (H); Acidic: Aspartic acid (D), Glutamic acid (E), Asparagine (N), Glutamine (Q); hydrophilic: Glycine (G), Alanine (A), Valine (V), Leucine (L), Isoleucine (I); Hydrophobic: Phenylalanine (F), Tyrosine (Y), Tryptophan (W); Sulfur-containing: Methionine (M), Cysteine (C). In addition, sequences that differ by conservative variations are generally homologous.

Descriptions of the molecular biological techniques useful to the practice of the invention including mutagenesis, PCR, cloning, and the like include Berger and Kimmel, GUIDE TO MOLECULAR CLONING TECHNIQUES, METHODS IN ENZYMOLOGY, volume 152, Academic Press, Inc., San Diego, Calif. (Berger); Sambrook et al., MOLECULAR CLONING—A LABORATORY MANUAL (2nd Ed.), Vol. 1-3, Cold Spring Harbor Laboratory, Cold Spring Harbor, N.Y., 1989, and CURRENT PROTOCOLS 1N MOLECULAR BIOLOGY, F. M. Ausubel et al., eds., Current Protocols, a joint venture between Greene Publishing Associates, Inc. and John Wiley & Sons; Inc.; Berger, Sambrook, and Ausubel, as well as Mullis et al., U.S. Pat. No. 4,683,202 (1987); PCR PROTOCOLS A GUIDE TO METHODS AND APPLICATIONS (Innis et al. eds), Academic Press, Inc., San Diego, Calif. (1990) (Innis); Arnheim & Levinson (Oct. 1, 1990) C&EN 36-47; Lueng, et al.

In yet another embodiment, a nucleic acid of the invention is expressed in mammalian cells using a mammalian expression vector. For suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

A polynucleotide can be a DNA molecule, a cDNA molecule, genomic DNA molecule, or an RNA molecule. A polynucleotide as DNA or RNA can include a sequence wherein T (thymidine) can also be U (uracil). If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are substantially complementary to each other when a sufficient number of corresponding positions in each molecule are occupied by nucleotides that can hybridize with each other in order to effect the desired process.

Transformation of a host cell with recombinant DNA may be carried out by conventional techniques as are well known to those skilled in the art. By “transformation” is meant a permanent or transient genetic change induced in a cell following incorporation of new DNA (i.e., DNA exogenous to the cell).

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the alpha-fetoprotein promoter (Campes and Tilghman, 1989. Genes Dev. 3: 537-546).

In any of the embodiments, the nucleic acids encoding the MIF, MIF binding protein, and/or MIF receptor can be present as: one or more naked DNAs; one or more nucleic acids disposed in an appropriate expression vector and maintained episomally; one or more nucleic acids incorporated into the host cell's genome; a modified version of an endogenous gene encoding the components of the complex; one or more nucleic acids in combination with one or more regulatory nucleic acid sequences; or combinations thereof. The nucleic acid may optionally comprise a linker peptide or fusion protein component, for example, His-Tag, FLAG-Tag, fluorescent protein, GST, TAT, an antibody portion, a signal peptide, and the like, at the 5′ end, the 3′ end, or at any location within the ORF.

In a preferred embodiment, the nucleic acid of the invention comprises a polynucleotide encoding the soluble (i.e., the extracellular) portion of a MIF receptor. Any of the embodiments described herein, can be achieved using standard molecular biological and genetic approaches well known to those of ordinary skill in the art.

Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂, RbCl, liposome, or liposome-protein conjugate can be used. Transformation can also be performed after forming a protoplast of the host cell or by electroporation. These examples are not limiting on the present invention; numerous techniques exist for transfecting host cells that are well known by those of skill in the art and which are contemplated as being within the scope of the present invention.

When the host is a eukaryote, such methods of transfection with DNA include calcium phosphate co-precipitates, conventional mechanical procedures such as microinjection, electroporation, insertion of a plasmid encased in liposomes, or virus vectors, as well as others known in the art, may be used. The eukaryotic cell may be a yeast cell (e.g., Saccharomyces cerevisiae) or may be a mammalian cell, including a human cell. For long-term, high-yield production of recombinant proteins, stable expression is preferred.

Polypeptides

In other embodiments, the invention pertains to isolated nucleic acid molecules that encode MIF, MIF binding proteins, and/or MIF receptor polypeptides, antibody polypeptides, or biologically active portions thereof. The polypeptides of the complex can be formed, for example, using a peptide synthesizer according to standard methods; or by expressing each polypeptide separately in a cell or cell extract, then isolating and purifying the polypeptide.

Antibodies

The term “antibody” as used herein refers to immunoglobulin molecules and immunologically active portions of immunoglobulin (Ig) molecules, i.e., molecules that contain an antigen-binding site that specifically binds (immunoreacts with) an antigen, comprising at least one, and preferably two, heavy (H) chain variable regions (abbreviated herein as VH), and at least one and preferably two light (L) chain variable regions (abbreviated herein as VL). Such antibodies include, but are not limited to, polyclonal, monoclonal, chimeric, single chain, Fab, Fab′ and F(ab′)2 fragments, and an Fab expression library. The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

Antibodies can be prepared from the intact polypeptide or fragments containing peptides of interest as the immunizing agent. A preferred antigenic polypeptide fragment is 15-100 contiguous amino acids of MIF, MIF binding protein, or MIF receptor protein. In one embodiment, the peptide is located in a non-transmembrane domain of the polypeptide, e.g., in an extracellular or intracellular domain. An exemplary antibody or antibody fragment binds to an epitope that is accessible from the extracellular milieu and that alters the functionality of the protein. In certain embodiments, the present invention comprises antibodies that recognize and are specific for one or more epitopes of a MIF protein, MIF binding protein, and/or MIF receptor protein, variants, portions and/or combinations thereof. In alternative embodiments antibodies of the invention may target and interfere with the MIF/MIF receptor interaction to inhibit signaling.

The preparation of monoclonal antibodies is well known in the art; see for example, Harlow et al., Antibodies: A Laboratory Manual, page 726 (Cold Spring Harbor Pub. 1988). Monoclonal antibodies can be obtained by injecting mice or rabbits with a composition comprising an antigen, verifying the presence of antibody production by removing a serum sample, removing the spleen to obtain B lymphocytes, fusing the lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by techniques well known in the art.

In other embodiments, the antibody can be recombinantly produced, e.g., produced by phage display or by combinatorial methods. Phage display and combinatorial methods can be used to isolate recombinant antibodies that bind to MIF, MIF binding proteins, and/or MIF receptor proteins or fragments thereof (as described in, e.g., Ladner et al. U.S. Pat. No. 5,223,409; Fuchs et al. (1991) Bio/Technology 9:1370-1372; Hay et al. (1992) Hum Antibod Hybridomas 3:81-85; Huse et al. (1989) Science 246:1275-1281; Clackson et al. (1991) Nature 352:624-628; Gram et al. (1992) PNAS 89:3576-3580.

Human monoclonal antibodies can also be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855).

A therapeutically useful antibody to the components of the complex of the invention or the complex itself may be derived from a “humanized” monoclonal antibody. Humanized monoclonal antibodies are produced by transferring mouse complementarity determining regions from heavy and light variable chains of the mouse immunoglobulin into a human variable domain, then substituting human residues into the framework regions of the murine counterparts. The use of antibody components derived from humanized monoclonal antibodies obviates potential problems associated with immunogenicity of murine constant regions. Techniques for producing humanized monoclonal antibodies can be found in Jones et al., Nature 321: 522, 1986 and Singer et al., J. Immunol. 150: 2844, 1993. The antibodies can also be derived from human antibody fragments isolated from a combinatorial immunoglobulin library; see, for example, Barbas et al., Methods: A Companion to Methods in Enzymology 2, 119, 1991. In addition, chimeric antibodies can be obtained by splicing the genes from a mouse antibody molecule with appropriate antigen specificity together with genes from a human antibody molecule of appropriate biological specificity; see, for example, Takeda et al., Nature 314: 544-546, 1985. A chimeric antibody is one in which different portions are derived from different animal species.

Anti-idiotype technology can be used to produce monoclonal antibodies that mimic an epitope. An anti-idiotypic monoclonal antibody made to a first monoclonal antibody will have a binding domain in the hypervariable region that is the “image” of the epitope bound by the first monoclonal antibody. Alternatively, techniques used to produce single chain antibodies can be used to produce single chain antibodies. Single chain antibodies are formed by linking the heavy and light chain fragments of the Fv region via an amino acid bridge, resulting in a single chain polypeptide. Antibody fragments that recognize specific epitopes, e.g., extracellular epitopes, can be generated by techniques well known in the art. Such fragments include Fab fragments produced by proteolytic digestion, and Fab fragments generated by reducing disulfide bridges. When used for immunotherapy, the monoclonal antibodies, fragments thereof, or both may be unlabelled or labeled with a therapeutic agent. These agents can be coupled directly or indirectly to the monoclonal antibody by techniques well known in the art, and include such agents as drugs, radioisotopes, lectins and toxins.

The dosage ranges for the administration of monoclonal antibodies are large enough to produce the desired effect, and will vary with age, condition, weight, sex, age and the extent of the condition to be treated, and can readily be determined by one skilled in the art. Dosages can be about 0.1 mg/kg to about 2000 mg/kg. The monoclonal antibodies can be administered intravenously, intraperitoneally, intramuscularly, and/or subcutaneously.

In certain embodiments of the invention, at least one epitope encompassed by the antigenic peptide is a region of MIF, a MIF binding protein, and/or a MIF receptor that is located on the surface of the protein, e.g., a hydrophilic region. A hydrophobicity analysis of the protein sequence will indicate which regions of a polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein. A protein of the invention, or a derivative, fragment, analog, homolog or ortholog thereof, may be utilized as an immunogen in the generation of antibodies that immunospecifically bind these protein components.

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein-Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol. 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology, 10:779-783 (1992)); Lonberg et al. (Nature, 368:856-859 (1994)); Morrison (Nature, 368:812-13 (1994)); Fishwild et al, (Nature Biotechnology, 14:845-51 (1996)); Neuberger (Nature Biotechnology, 14:826 (1996)); and Lonberg and Huszar (Intern. Rev. Immunol., 13:65-93 (1995)).

Human antibodies may additionally be produced using transgenic nonhuman animals which are modified so as to produce fully human antibodies rather than the animal's endogenous antibodies in response to challenge by an antigen. The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096.

Fab Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of Fab expression libraries (see e.g., Huse, et al., Science 246:1275-1281 (1989)) to allow rapid and effective identification of monoclonal Fab fragments with the desired specificity for a protein or derivatives, fragments, analogs or homologs thereof. Antibody fragments that contain the idiotypes to a protein antigen may be produced by techniques known in the art including, but not limited to: (i) an F(ab′)2 fragment produced by pepsin digestion of an antibody molecule; (ii) an Fab fragment generated by reducing the disulfide bridges of an F(ab′)2 fragment; (iii) an Fab fragment generated by the treatment of the antibody molecule with papain and a reducing agent and (iv) Fv fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit. Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. Similar procedures are disclosed in WO 93/08829, published May 13, 1993, and Traunecker et al., EMBO J., 10:3655-3659 (1991).

Antibody variable domains with the desired binding specificities (antibody-antigen combining sites) can be fused to immunoglobulin constant domain sequences. For further details of generating bispecific antibodies see, for example, Suresh et al., Methods in Enzymology, 121:210 (1986); and Brennan et al., Science 229:81 (1985).

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)2 molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The “diabody” technology described by Hollinger et al., Proc. Natl. Acad. Sci. USA 90:6444-6448 (1993) has provided an alternative mechanism for making bispecific antibody fragments. Another strategy for making bispecific antibody fragments by the use of single-chain Fv (sFv) dimers has also been reported. See, Gruber et al., J. Immunol. 152:5368 (1994). Antibodies with more than two valencies are contemplated. For example, trispecific antibodies can be prepared. Tutt et al., J. Immunol. 147:60 (1991). Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA.

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Immunoconjugates

The invention also pertains to immunoconjugates comprising an antibody conjugated to a chemical agent, or a radioactive isotope (i.e., a radioconjugate). Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257: 286-288 (1982) via a disulfide-interchange reaction.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 500 mg/kg body weight.—Common dosing frequencies may range, for example, from twice daily to once a week.

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York. The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions. The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., Fab or F(ab)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that: it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Thory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques intracavity, or transdermally, alone or with effector cells.

Preparations for administration of the therapeutic of the invention include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Vehicles include sodium chloride solution, Ringer's dextrose, dextrose and sodium chloride, lactated Ringer's intravenous vehicles including fluid and nutrient replenishers, electrolyte replenishers, and the like. Preservatives and other additives may be added such as, for example, antimicrobial agents, anti-oxidants, chelating agents and inert gases and the like.

The compounds, nucleic acid molecules, polypeptides, and antibodies (also referred to herein as “active compounds”) of the invention, and derivatives; fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, intraperitoneal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor™. (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., the therapeutic complex of the invention) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups, or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles. (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring, and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethan-e, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents. Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. The pharmaceutical compositions can be included in a container, pack, or dispenser together with instructions for administration.

A therapeutically effective dose refers to that amount of the therapeutic sufficient to result in amelioration or delay of symptoms. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Also disclosed according to the present invention is a kit or system utilizing any one of the methods, selection strategies, materials, or components described herein. Exemplary kits according to the present disclosure will optionally, additionally include instructions for performing methods or assays, packaging materials, one or more containers which contain an assay, a device or system components, or the like.

Additional objects and advantages of the present invention will be appreciated by one of ordinary skill in the art in light of the current description and examples of the preferred embodiments, and are expressly included within the scope of the present invention.

### EXAMPLES

Animal care. Mice lacking MIF were generated by homologous recombination and were then backcrossed into a pure C57BL/6 background. Animals were bred and maintained at the University of Connecticut Health Center for Laboratory Animal Care under National Institutes of Health guidelines. All procedures were approved by an institutional animal care committee. All surgeries were performed using 2 month old female wild-type (WT) and mif −/− knockout (K/O) mice. Mice had ad lib access to water and food (Teklad 2918; Harlan, Indianapolis).

Animal surgery. Partial bladder outlet obstruction (pBOO) surgery was created as described by Lemack et al. and by Felsen et al. Anesthesia was maintained using inhaled Isoflurane delivered at 1.5-2% in pure oxygen flow (250 ml/min). A 22-gauge angiocatheter was introduced into the bladder through the urethra under sterile conditions. A 1-cm lower mid-line incision was made. Blunt dissection allowed for identification of the urethra with angiocatheter in place. After minimal dissection of surrounding tissues, a curved clamp was passed posteriorly and a 4-0 silk tie secured snuggly but not tightly at the bladder neck. If too much force was needed to remove the catheter (too tight) or if there was no movement of periurethral tissue (too loose), the suture was replaced. Sham-operated mice underwent an identical procedure, but the tie was not placed. Animals were observed for signs of discomfort for the duration of the study. Buprenorphine (0.1 mg/kg) was injected subcutaneously for post-operative pain control. All pBOO animals were sacrificed 3 weeks following surgery.

RNA extraction and quantitative real-time PCR. Total RNA was extracted from murine bladder tissues with TRIzol Reagent (Invitrogen Life technologies, Carlsbad, Calif.) following the manufacturer's protocol. Samples were treated with DNase I (DNA-Free™ kit, Ambion, Austin, Tex.) to remove contaminating DNA from RNA preparations following the manufacturer's protocol. Total RNA was converted to cDNA by ABI High Capacity cDNA Archive Kit (Applied Biosystems, Foster City, Calif.) following the manufacturer's protocol. Real-Time PCR was performed for different gene expression in separate wells (singleplex assay) of 96-well plates, in a reaction volume of 20 μl. GAPDH was used as endogenous control. Three replicates of each sample were amplified using Assays-on-Demand Gene Expression assay (Assay# Mm00478374_m1 for COX2, Mm00443258_m1, Mm01604696_g1 for MIF and Mm99999915_g1 for GAPDH) which contained predesigned unlabeled gene-specific PCR primers and TaqMan MGB FAM dye-labeled probe. The PCR reaction mixture (including 2× TaqMan Universal PCR Master Mix, 20× Assays-on-Demand Gene Expression Assay Mix, 50 ng of cDNA) was run in Applied Biosystems ABI Prism 7300 Sequence Detection System instrument utilizing universal thermal cycling parameters.

All primers were tested for equal efficiency over a range of target gene concentration before use. The relative quantification of target gene expression in a test sample to a reference calibrator sample (ΔΔCt Method) was used for data analysis.

Tissue preparation for trichrome staining and immunohistochemistry. Following sacrifice, bladders were harvested and bivalved in a reproducible manner across the detrusor mid-section. Specimens were then fixed in 4% paraformaldehyde-PBS and embedded in paraffin wax with the detrusor mid-section acing and lying parallel to the microtome blade. Twenty coronal sections (5 μm thickness) were obtained from each detrusor mid-section and mounted on slides. This consisted of 4 sets of sequential sections with each set separated by 50 μm. A single section from each set was randomly chosen for Masson's trichrome staining and subsequent quantitative analysis. After dewaxing and hydration, slides first underwent treatment in Bouin solution (Poly Scientific, Bay Shore, N.Y.) for 1 hr at 56° C. They were then serially stained in Weigert hematoxylin and Gomori trichrome stain (Poly Scientific) and differentiated in 0.5% acetic acid before going through dehydration and mounting with Cytoseal. This protocol stains muscle cell cytoplasm red, collagen blue and nuclei purple.

Quantitative morphometric analysis. As described above, four sections randomly selected from different regions of ladder mid-section underwent quantitative morphometric analysis. Slides were viewed in a blinded fashion. Digital micrographs were taken using a Zeiss Axioshop 2 Plus microscope. Images were captured and separated into urothelial, suburothelial and muscle compartments. Images from the muscle compartment were then digitized into red and blue signal representing muscle and collagen, respectively and were then quantified using Adobe© and Image J software (http://rsb.info.nih.gov/ij/). An eye objective-mounted grid was placed over 10 distinct regions of each section to count numbers of grid intercepts overlying detrusor tissue, muscle and muscle nuclei. Results were then analyzed as: 1) the collagen to muscle ratio; 2) amount of signal representing muscle per bladder mid-section; 3) amount of signal representing collagen per bladder mid-section and 4) numbers of nucleated muscle cells per bladder mid-section.

Immunohistochemistry. Mounted slides were prepared and deparaffinized as noted above. Endogenous peroxidase was quenched with 3% H₂O₂. Epitope retrieval was performed using a Power Block Universal Blocking Agent (Biogenex, San Ramon, Calif.) and non-specific staining was blocked with normal goat serum. Tissue sections were incubated with a polyclonal anti-MIF antibody (CPM300; Cell Sciences, Canton, Mass.) at 4° C. overnight. This was followed by washes, incubations with an appropriate biotinylated secondary antibody, DAB development (Vector Labs, Burlingame, Calif.), a Methyl Green counterstain, dehydration and mounting. Cultured cells were stained using a α-smooth muscle actin mouse monoclonal (A2547; Sigma, St Louis, Mo.) and a pan-cadherin rabbit polyclonal (C 3678; Sigma) antibody. Monoclonal antibody was visualized with CY3-tagged goat anti-mouse IgG (H+L) (#115-165-146; Jackson Immunoresearch Laboratories, West Grove, Pa.), while the polyclonal antibody was visualized with Alexa 488-tagged goat anti rabbit F(ab) immunoglobulin G (H+L) (# A11070; Molecular Probes, Carlsbad, Calif.). Cells were viewed and confocal images obtained using a Zeiss LSM 410 confocal microscope.

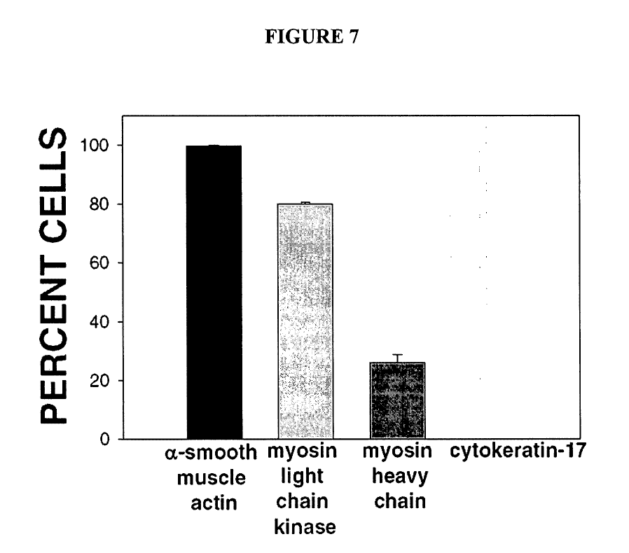

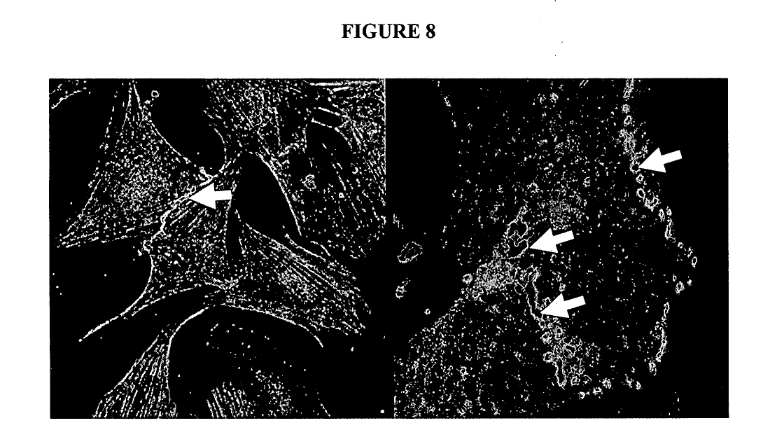

Tissue culture. Primary bladder muscle cultures were derived from newborn Sprague-Daley rats (Charles River; Wilmington, Mass.). The bladders were harvested, minced and added to 3 ml dissociation solution containing 0.5 mg/ml collagenase, 0.5 mg/ml elastase, 2 μg/ml DNAse (Worthington, Lakewood, N.J.) and 1 mg/ml soybean trypsin inhibitor (Gibco, Carlsbad, Calif.). The tissue was triturated at 15 minute intervals for 45 minutes using fire polished pasteur pipets. The cell solution was filtered through a 40 μm cell strainer and spun at 1000 rpm for 5 minutes. The supernatant was aspirated and cells resuspended in DMEM-F/12 medium containing 1% FBS, 1% N-2 supplement and 1% Penicillin/Streptomycin (Gibco). The cell number was determined and cells were plated at a density of 25,000 cells/cm2 in poly-L-lysine coated (P-6282, Sigma) 12-well plates (#3513, Corning, Corning, N.Y.) for biochemical and flow cytometry studies and on plastic 8-well chamber slides (#354108, BD Bioscience, San Jose, Calif.) for immunohistochemistry or TUNEL staining. Cells were maintained at 37° C. and 5% CO₂ in a humidified chamber. The presence of 1% FBS was required for the success of initial plating. However, 24 hours after plating cells were maintained in serum-free defined medium in order to promote the presence of the fully differentiated contractile phenotype. Cell labeling studies established that essentially all of our cultured cells expressed smooth muscle markers, with no apparent contamination by urothelial cells (FIG. 7). Treatments took place on day 5 after plating when in our system most cultured cells express smooth muscle differentiation markers, with many making cadherin-positive cell-cell contact (FIG. 8).

Tissue culture characterization. Flow cytometry was used to evaluate the expression of both bladder muscle and urothelial differentiation markers. Floating and trypsinized adherent cells were separated into equal aliquots and were labeled with monoclonal antibodies for MLCK (M7905, Sigma), calponin (C2687, Sigma), MHC (SC-6956, Santa Cruz, Santa Cruz, Calif.) or cytokeratin-17 (M7046, Dako, Carpinteria, Calif.). Following incubation with a PEconjugated goat anti-mouse antibody, cell aliquots were analyzed on flow Cytometry as described below.

MIF ELISA. A sandwich ELISA utilizing a monoclonal IgG1 and a purified polyclonal IgG has been extensively validated. MIF concentrations were calculated by extrapolation from a quadratic standard curve using human rMIF (range: 0-12 ng/ml, sensitivity: 150 pg/ml).

Apoptosis detection. Flow cytometry was also used for detection of apoptosis. Treatments were performed using mouse rMIF which differs from rat MIF by one amino acid and that does not affect protein bioactivity. Recombinant mouse MIF was produced in our laboratory, and purified free from endotoxin as described previously. Following treatment with rMIF, cells were analyzed using a kit (PF032, Calbiochem, San. Diego, Calif.) to separate apoptotic and necrotic cells on flow cytometry. Both floating and trypsinized adherent cells were collected and washed in PBS. Binding buffer (0.5 ml), 10 μl Media Binding Reagent and 1.25 μl of Annexin V-FITC was added to the cells. This solution was incubated for 15 minutes at room temp. Cells were spun down and resuspended in binding buffer (0.5 ml) and 10 μl propidium iodide. Cells were analyzed using a Becton Dickenson FACS caliber flow cytometer with collection and analysis of data performed using Becton Dickinson CELLQuest software. TUNEL staining was detected using a kit (Roche) with DAB microscopy of cells grown 5-7 days in 8-well chamber slides. To quantify the rate of apoptosis (TUNEL stained cells/all visible cells), we counted cells in 4 randomly chosen fields in each of 4 wells per group. All studies were performed in the presence of cyclohexamide (1 μg/ml; C4859, Sigma) in all of the treatment and control wells. This inhibitor of protein synthesis has been shown to enhance the toxicity of TNF-α in human saphenous vein smooth muscle cells, yet at these concentrations cyclohexamide alone does not induce apoptosis or necrosis either in vascular or in bladder (data not shown) smooth muscle cells.

Statistical analysis. Statistical comparisons were performed using SigmaStat (San Rafael, Calif.) and SPSS (Chicago, Ill.) software.

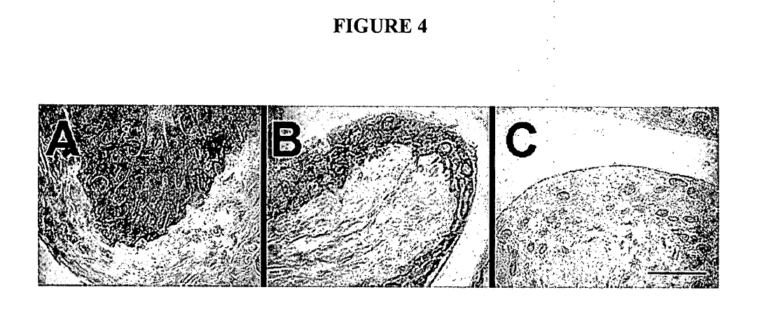

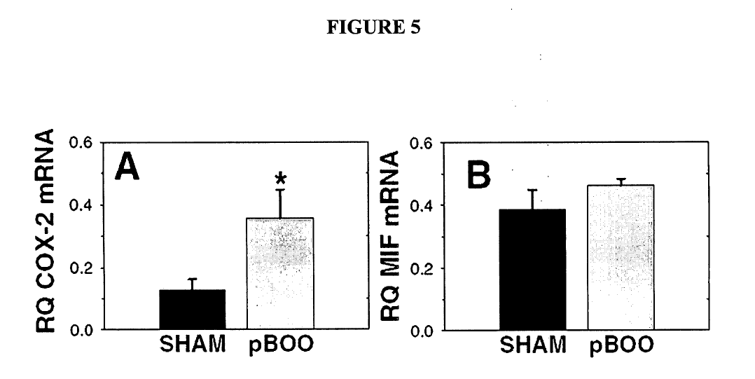

MIF expression. In sham-operated WT mice, intense cytoplasmic, and occasionally nuclear, MIF immunoreactivity was detected throughout basal and apical layers of the urothelium (FIG. 4A). After 3 weeks of pBOO, MIF immunoreactivity was markedly decreased in all urothelial cells (FIG. 4B), but increased in very few muscle or mononuclear inflammatory cells (not shown). In MIF K/O mice, MIF immunoreactivity was completely absent (not shown). At this 3 week time point following pBOO, overall mif mRNA levels remained similar in pBOO and sham-operated WT bladders (FIG. 5). However, as expected from results of earlier studies, pBOO increased cox-2 mRNA levels more than twofold.

Body and bladder weight. Body weights of sham-operated WT and MIF K/O mice were similar, with no change following pBOO (Table 4). Whole bladder weights were 36.7% higher in sham-operated MIF K/O mice and 50.8% higher in obstructed MIF K/O mice as compared to sham-operated WT controls (Table 4). While pBOO surgery appeared to increase bladder weights in some WT and some MIF KO mice, overall these differences did not reach statistical significance. Expressing bladder weights per body weight did not change these relationships (Table 4).

Table 4. Body and whole bladder weights in WT and MIF K/O mice 3 weeks after sham or pBOO surgery. Results are presented as means +/−standard deviation. Values were analyzed using one-way analysis of variance and, when significant, post hoc multiple comparisons were performed using the Holm-Sidak method. Statistical comparisons were adjusted for multiple comparisons. Statistically significant differences as compared to WT sham animals (*) are indicated in the table. The experiment-wise Type I error rate was set at α=0.05.

| | Wild-Type | Wild-Type | MIF K/O | MIF K/O |
|---|---|---|---|---|
| Group (n) | sham (11) | pBOO (10) | sham (10) | pBOO (9) |
| Body | 21.5 ± 1.8 | 21.9 ± 1.0 | 21.8 ± 1.4 | 20.7 ± 1.5 |
| Weight | | | | |
| (gms) | | | | |
| Bladder | 19.9 ± 2.9 | 24.5 ± 9.5 | 27.2 ± 3.9* | 30.0 ± 8.0* |
| Weight | | | | |
| (mgs) | | | | |
| Bladder | 0.93 ± 0.16 | 1.12 ± 0.44 | 1.25 ± 0.20* | 1.45 ± 0.37* |
| Weight/ | | | | |
| body | | | | |
| weight | | | | |
| (mgs/gms) | | | | |

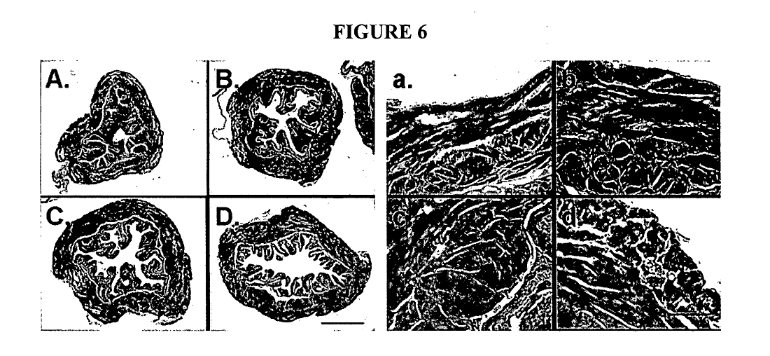

Detrusor morphology. Using Masson's trichrome stain, muscle cytoplasm is red, collagen blue and nuclei purple. At a low magnification, bladders from MIF K/O sham-operated mice (FIG. 6C) appeared somewhat larger than those from sham-operated WT (FIG. 6A) mice, yet their gross appearance was similar. Following 3 weeks of pBOO, WT bladders seemed slightly larger (FIG. 6B) with a greater degree of fibrosis (dark stain) at a higher magnification in pBOO (FIG. 6b) as compared to sham-operated (FIG. 6A,a) mice. In contrast, pBOO surgery did not appear to increase the size of bladders in MIF K/O mice (FIG. 6D), with similar degrees of fibrosis in pBOO (FIG. 6d) and sham-operated (FIG. 6c) MIF K/O mice.

Detrusor quantitative morphometry. All of the following quantitative morphometric measurements were performed in a total of 4 sections randomly-selected from the mid-detrusor region of each bladder (Table 5). A two-way repeated measures ANOVA model was used for statistical analysis. A log transformation was applied to the collagen, as well as the collagen to muscle ratio variables in order to correct for skewness and non-constant variance. No transformation was deemed appropriate for the muscle and the nucleated muscle cell count variables. The estimated between mice and within mice (due to sections) variances were: Log (collagen to muscle ratio): Var_{mice}=0.06124, Var_{section}=0.07076; Log (collagen): Var_{mice}=0.03981, Var_{section}=0.06490; Muscle: Var_{mice}=6.1277×10₈, Var_{section}=10.099×10₈ Nucleated Muscle Cell Count: Var_{mice}=3823, Var_{section}=244991. A statistically significant interaction between the K/O (versus WT) factor and the pBOO (versus sham) factor was detected for the collagen to muscle ratio variable (p=0.0167), the collagen variable (p=0.0216) and the nucleated muscle cell count variable (p=0.0013). No similar statistically significant interactions were detected for the muscle variable (p=0.4283). However, the MIF WT versus MIF K/O comparison was statistically significant for muscle only (p=0.0001). All the reported p-values and 95% confidence intervals (Table 5) were adjusted for multiple comparisons using the Tukey-Kramer method.

Table 5. Impact of pBOO and MIF on mid-detrusor quantitative morphometric measurements. Mid-detrusor sections underwent trichrome staining, with four sections then randomly selected for quantitative morphometric analysis as described in MATERIALS AND METHODS. Wild-type and MIF K/O female mice were studied 3 weeks after sham or pBOO surgery, with 9-11 animals in each experimental group (see above). The mid-detrusor collagen to muscle ratio and collagen are reported as median plus 95% confidence intervals since a log transformation was used in the analysis for these measures. In contrast, mid-detrusor midsection muscle and nucleated muscle cell counts are reported as mean plus 95% confidence intervals. Statistical comparisons were adjusted for multiple comparisons. Statistically significant differences as compared to WT sham animals (*) or as compared to WT pBOO animals (@) are indicated in the table. The experiment-wise Type I error rate was set at α=0.05.

| | Wild-Type sham | Wild-Type pBOO | MIF K/O sham | MIF K/O pBOO |
|---|---|---|---|---|
| Group (n) | (11) | (10) | (10) | (9) |
| Collagen to | 0.245 | 0.466* | 0.317^{@} | 0.386* |
| muscle ratio | [0.206-0.291] | [0.389-0.558] | [0.265-.0380] | [0.319-0.467] |
| Collagen (mm²) | 0.208 | 0.373* | 0.323^{@} | 0.402* |
| | [0.180-0.241] | [0.320-0.434] | [0.277-0.375] | [0.342-0.473] |
| Muscle (mm²) | 0.868 | 0.816 | 1.040*^{@} | 1.059*^{@} |
| | [0.784-0.951] | [0.729-0.904] | [0.953-0.1.128] | [0.966-1.151] |
| Muscle Cell | 1,629 | 1,272* | 1,570 | 1,866^{@} |
| Count | [1,450-1,807] | [1,085-1,460] | [1,382-1,758] | [1,661-2,701] |

No statistically significant effect on the mid-detrusor collagen to muscle ratio was observed when rendering mice null for the mif gene [CI: 0.93-1.81; p=0.17]. As noted above (Table 4), MIF K/O bladders were nearly 40% heavier than WT bladders. In MIF K/O mice median mid-detrusor collagen was 1.6-fold [CI: 1.17-2.05; p=0.001] higher and mean mid-detrusor muscle was 0.173 mm2 greater [CI: 0.012-0.333; p=0.0305] (an observed 19.9% relative increase) when compared to wild-type animals. The median of the mid-detrusor collagen to muscle ratio was 1.9-fold [CI for the ratio of medians: 1.37-2.65; p<0.0001] higher in obstructed as compared to sham-operated wildtype animals (Table 5). In contrast, in mice rendered null for the mif gene, pBOO surgery had no impact on the collagen to muscle ratio [CI: 0.86-1.72; p=0.44]. The median mid-detrusor collagen was 1.8-fold [CI: 1.35-2.37; p<0.0001] higher in pBOO as compared to sham-operated WT mice (Table 5). In contrast to its effect in WT animals, pBOO had no statistically significant effect on mid-detrusor collagen in MIF K/O mice [CI: 0.93-1.67; p=0.20]. A mif gene deletion increased mean mid-detrusor muscle by 0.242 mm2 [0.073-0.411; p=0.0025] (an observed 29.7% relative increase) in pBOO mice. Following pBOO, the mean mid-detrusor nucleated muscle cell count decreased by 356.04 [CI: 11.87-700.22; p=0.04] (an observed 21.9% relative decrease) in WT mice, while in the absence of MIF, the mean mid-detrusor nucleated muscle cell count in obstructed mice was not statistically significantly different than that of sham-operated controls [CI: −73.53-665.15; p=0.155]. Finally, the observed mean mid-detrusor nucleated muscle cell count for obstructed MIF K/O mice was higher by 593.39 than the mean for obstructed WT mice [CI: 224.04-871.36; p=0.0006] (an observed 46.7% relative increase).

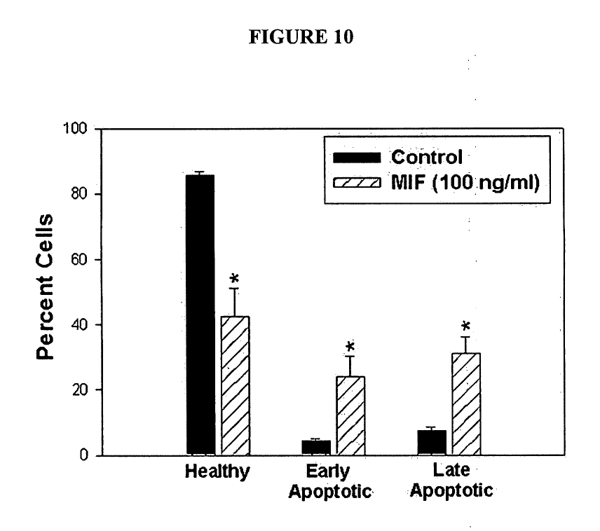

MIF effects on detrusor muscle cells in vitro. All tissue culture studies were performed 5 days after plating. Immunolabeling with antibodies for smooth muscle cell and urothelial markers followed by flow cytometry (FIG. 7A) revealed that nearly all (99.7%±0.1%) of cells express α-smooth muscle actin, 80.0±0.6% express myosin light chain kinase (MLCK), 26.0%±3% express myosin heavy chain (MHC) and none express urothelial markers such as cytokeratin-17. Moreover, images obtained using confocal microscopy revealed that many cultured cells expressing α-smooth muscle actin make cadherin-positive cell-cell contact (FIG. 8) similar to the “zipper”-like areas of contact described between epithelial cells. Treatment of these cultures with TNF-α (50 ng/ml) for 24 hours resulted in an approximately 30% increase in MIF protein levels detectable in the supernatant (p<0.01; FIG. 7B). Treatment with rMIF protein (100 ng/ml) over 3 hours increased the proportion of TUNEL-positive bladder muscle cells in our cultures more than 2.5-fold (7.8% vs 20.1; p<0.05; FIG. 6). The addition of rMIF protein (100 ng/ml for 24 hours) also decreased the proportion of healthy cells, while increasing the proportion of cells demonstrating evidence of early apoptosis or late apoptosis/necrosis (FIG. 10).

Macrophage migration inhibitory factor (MIF) is abundantly expressed in urothelial cells and is known to be released by varied inflammatory stimuli. MIF has been shown to enhance fibroblast survival. We formulated a hypothesis proposing that MIF could play a physiological role as a mediator of smooth muscle death and collagen deposition in the obstructed bladder. Mice rendered null for the mif gene (MIF K/O) provide an optimal model to examine this hypothesis since they are viable, fertile and exhibit no apparent phenotype under basal conditions. Our interest was also prompted by the recent description of functional polymorphisms in the human MIF gene, which are being shown to be associated with different chronic inflammatory conditions. Using existing protocols for creating pBOO in mice, we identified a time point at which two of the key morphologic features of detrusor underactivity (detrusor muscle cell loss and collagen deposition) become evident in a reproducible fashion in our obstructed wild-type mice. In order to perform stereologically-sound quantitative morphometric measurements in a 3-dimensional structure such as the bladder, we adapted approaches developed by scientists in other fields.

Obstruction studies in mice. The use of genetically-modified mice offers a unique opportunity to implicate a specific gene in the sequence of events by which partial bladder obstruction results in specific categories of detrusor responses. Several investigators have pioneered the development of female and male mouse models of pBOO. Unfortunately, the impact of pBOO on detrusor structure has not always been consistent. For example, Lemack et al. studied young (4-6 m. old) ICR strain female mice and reported that 3 weeks after surgery the collagen to muscle ratio decreased by nearly a half from 1.38 in sham-operated to 0.72 in pBOO mice. In contrast, Felsen et al. used older middle-aged (8-12 m. old) C57BL6 female mice and reported a significant increase at the same time point, with the collagen to muscle ratio more than doubling from 0.72 in sham-operated to 1.5 in pBOO animals. Impact on bladder weights has also been variable. For example, bladder weights of our obstructed wild-type mice underwent only minimal (23%) and statistically non-significant increases (Table 5), resembling a similar (28%) increase described at this time-point by Lemack et al. In contrast, other investigators have reported a doubling in mouse bladder weights at 1 week and a near 3-fold increase at 5 weeks after obstruction. It remains to be seen to what extent differences in surgical technique, animal age, genetic factors, the selection of specific inbred mouse strains and/or “tightness” of the pBOO lesion are responsible for differences between these two studies. Our obstructed mice demonstrated evidence of elevated collagen to muscle ratio (Table 5) and raised cox-2 mRNA level (FIG. 5A), with a remarkably low degree of variability between individual mice in each of the obstructed groups. While none of these considerations detract from the validity and importance of our findings, it will be important for bladder researchers to develop tools to better standardize the “tightness” of pBOO lesions between different surgeons and laboratories.

Insights from other animal models. Studies evaluating detrusor responses to obstruction in rabbits have been much more numerous. For example, investigators have described hypertrophic changes involving muscle cells, followed by myocyte and axonal degeneration plus collagen deposition. ₃H-thymidine incorporation studies have demonstrated consistently increased incorporation of this label into urothelial and connective tissue compartments, but not into the muscularis layer. Based on these considerations, it has been proposed that most of the initial increase in overall bladder mass following obstruction can likely be attributed to growth involving individual muscle cells (muscle hypertrophy), with muscle cell death playing a likely role in the subsequent detrusor decompensation. Nevertheless, it has been very difficult to obtain in vivo evidence of apoptosis in pBOO, with published evidence limited to rabbit bladders “regressing” after removal of obstruction and to fetal ovine bladders obstructed in utero. Moreover, to date, no published study has examined the impact of pBOO on numbers of nucleated detrusor muscle cells. Unfortunately, quantitative morphometry is both time- and labor-intensive. Another obstacle to further progress, has been a failure to adapt the type of stereologically sound morphometric approaches used in fields such as neuroscience. Unlike other tissues (e.g. gut, bronchial airways), mammalian bladder smooth muscle cells lack a predictable organizational pattern. The obstructed detrusor also undergoes extensive structural plasticity and any quantitative morphometry protocol must respect basic stereological principals in order to avoid possible biases. The traditional collagen to muscle ratio provides only limited information since it does not establish the magnitude, or even the direction, of change involving either compartment. Moreover, in the absence of formal cell counting, no conclusions can be drawn about whether a change in overall muscle bulk results from differences in numbers of individual muscle cells and/or their size. Finally, basing quantitative morphometric measurements on a single tissue section presents substantial risks in terms of confounding experimental results with stereological biases.

MIF: A urothelium-derived mediator of bladder inflammation. Our studies indicate that, as shown by others, urothelium represents a rich source of MIF protein (FIG. 4A). As expected, cox-2 mRNA levels are upregulated following obstruction (FIG. 5A). Nevertheless, overall bladder mif mRNA levels remain unchanged (FIG. 5B), while MIF immunoreactivity is significantly decreased (FIG. 4B). Based on these findings, as well as studies examining the role of inflammatory signals in mediating MIF protein release from urothelial cells, macrophages and uterine epithelial cells, we believe that during pBOO preformed MIF protein undergoes release from urothelial cells and that regulation of this release represents a principal locus of regulation of MIF bioactivity in this system.

Impact of mif gene deletion on the mouse detrusor. As expected, rendering the mice null for the mif gene did not change body weight (Table 4). However, bladders from MIF K/O female mice were nearly 40% heavier than those from age-matched wild-type animals (Table 4). Based on our quantitative morphometric measurements (Table 5), increases in both muscle and connective tissue compartments in the mid-detrusor regions appear to be contributing to this increase in bladder mass. We believe that this represents the first evidence demonstrating an impact of a mif gene deletion on any animal phenotype under basal conditions.

Impact of partial obstruction on detrusor structure in wild-type mice. In our model system, 3 weeks of pBOO resulted in a 1.9-fold increase in the collagen to muscle ratio in the mid-detrusor region (FIG. 6; Table 5). This was accompanied by a 1.8-fold increase in collagen and no difference in muscle, but a 21.9% decrease in numbers of nucleated muscle cells in the mid-detrusor region (Table 5). We had selected this time point for our studies since we believe that these findings are indicative of early cellular events ultimately leading to detrusor underactivity. Our observation of decreased numbers of nucleated muscle cells together with no change in overall muscle mass most likely indicates the loss of individual muscle cells which is accompanied by a compensatory hypertrophy involving remaining muscle cells. Our preliminary electron microscope studies (not shown) support these observations with evidence of muscle and axonal degeneration, fibrosis, as well as enlarged hypertrophic muscle cells in wild-type mice studied 3 weeks after pBOO surgery.

Impact of mif gene deletion on detrusor responses to partial obstruction. Rendering mice null for the mif gene abolished increases in the collagen to muscle ratio and overall mid-detrusor collagen which had been demonstrated in wild-type mice after obstruction (FIG. 6; Table 5). Moreover, the presence of a mif gene deletion also abolished declines in mid-detrusor nucleated muscle counts which took place in wildtype mice after pBOO (Table 5). Mid-detrusor nucleated muscle counts were nearly 50% higher when comparing obstructed MIF K/O mice to obstructed wild-type mice (Table 5). In summary, our studies indicate that MIF must play an in vivo role in contributing to both detrusor fibrosis and muscle cell loss following bladder outlet obstruction.

MIF: a mediator of bladder fibrosis and muscle death in vitro. Published in vitro studies have demonstrated MIFs anti-apoptotic properties in macrophages, neutrophils and fibroblasts. While the impact of MIF on muscle survival or apoptosis has remained unknown, the addition of rMIF to differentiated L6 rat skeletal myotubes resulted in an unexpected increase in glycolysis with lactate accumulation. More recently, early MIF neutralization was shown to decrease endotoxin-mediated cardiac muscle cell death. With these considerations in mind, we explored the impact of adding a physiologic concentration of rMIF protein on parameters reflecting apoptosis in primary rodent bladder muscle cultures. In view of our hypothesis that in contrast to its anti-apoptotic effect in fibroblasts, MIF promotes apoptosis in bladder smooth muscle cells, our cultures were grown under serum-free conditions in order to ensure that cultured cells were more representative of the “contractile” phenotype typical of bladder muscle cells in vivo, as opposed to the “synthetic” fibroblast-like phenotype which is promoted by the addition of serum. As shown in FIG. 7, in our system nearly all (99.7%) cultured cells expressed α-smooth muscle actin, a classical, but relatively stable marker of smooth muscle differentiation, with no cells expressing the epithelial marker cytokeratin-17 which has been used to define urothelial cell cultures. Myosin light chain kinase (MLCK) and myosin heavy chain (MHC) represent markers of smooth muscle differentiation which are expressed in later stages of smooth muscle differentiation and appear to be much less stable or more sensitive to de-differentiation stimuli than is the case for α-smooth muscle actin. Nevertheless, approximately 80% of cultured cells expressed MLCK, while 26% expressed MHC. Recent studies have shown that the cellular phenotype within populations of cultured human vascular smooth muscle cells determines whether TNF-α induces a proliferative or a pro-apoptotic response. Serum-based bladder muscle culture protocols have proven remarkably useful in bladder research. Nevertheless, given the fact that bladder smooth muscle cells can undergo significant changes in cellular phenotype with development, aging, obstruction and the menopause, future studies will need to explore the influence of bladder muscle differentiation on cellular responses to relevant cytokines.

Mechanism of MIF-mediated smooth muscle toxicity. MIF concentrations of 50-100 ng/ml have been shown to result in a sustained induction of the extracellular-signal regulated kinase 1/2 (ERK 1/2) mitogen-activated protein kinase (MAPK). This effect has been shown to be dependent on MIF interacting with the membrane protein CD74. Activation of cell surface CD74 may require an interaction with CD44, an adhesion molecule also known to function as a receptor for hyaluronan (HA) and osteopontin. Both CD74 and CD44 have been shown to be expressed in rat urothelium, while CD44 mRNA and protein is also expressed in rat bladder muscle cells. Substance P induced bladder inflammation has been shown to upregulate both CD74 and CD44 protein expression. CD44 expression is increased in urothelium and interstitial bladder space following pBOO. It remains to be seen whether differences in CD74 and CD44 receptor expression or in downstream signaling pathways could account for the opposing effect of MIF on fibroblast and muscle cell survival. It has been shown that higher MIF concentrations (e.g. MIF>100 ng/ml) as could be achieved during inflammatory states, inhibit the JNK/AP-1 pathway through JAB1 activation. Recent studies indicate that a more transient and rapid ERK MAPK activation requires Src kinase activity, while Rho kinase appears to be involved in the pathways downstream from more sustained ERK MAPK activation.

In summary, applying stereologically-sound quantitative morphometric approaches to partially obstructed bladders from mice rendered null for the mif gene we are able to directly implicate, for the first time, a specific molecule in the cellular events leading to structural changes which define the presence of idiopathic detrusor underactivity, as well as detrusor underactivity in the setting of partial obstruction. Our data implicating MIF in obstruction-mediated muscle loss and fibrosis are supported by experiments which demonstrate that, contrary to MIFs established anti-apoptotic effects in fibroblasts, this cytokine is capable of pro-apoptotic effects in bladder smooth muscle cells. Human biopsy studies have clearly established the presence of muscle loss, fibrosis and axonal degeneration as highly reliable and early markers of detrusor underactivity. In fact, in some individuals such ultrastructural changes may precede the presence of urodynamically-detectable detrusor underactivity. Nevertheless, given advances in our ability to conduct reliable urodynamics in mice, it will be important to similarly link these structural changes to altered detrusor performance in mice.

MIF likely has effects on the extracellular matrix since MIF has been shown to induce MMP-9 in vascular smooth muscle cells and macrophages, MMP-9 and -13 in osteoblasts, as well as MMP-1 and MMP-3 in fibroblasts. Induction of such enzymes may destabilize human atherosclerotic plaques and could contribute to some of the degenerative changes involving both cells and extracellular matrix in DU.

The human mif gene promoter expresses several functional polymorphisms which have been shown to correlate with MIF protein expression and disease severity in several inflammatory conditions. By treating with an antagonist of MIF release or biological activity we can modulate the progression of common inflammatory conditions.

It is understood that the examples and embodiments described herein are for illustrative purposes only and that various modifications or changes in light thereof will be suggested to persons skilled in the art and are included within the spirit and purview of this application and are considered within the scope of the appended claims.

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims.

1. A method of treating or preventing a bladder-related disorder in an organism comprising the step of administering an effective amount of an antagonist or an inhibitor of at least one of MIF expression, MIF release, MIF activity or MIF receptor binding to an organism in need thereof.

2. The method of claim 1, wherein the antagonist or inhibitor is at least one member selected from the group consisting of a chemical compound comprising an isoxazoline moiety, an antagonist of a MIF receptor, an antibody to a MIF polypeptide, an antibody to a MIF receptor polypeptide, an enzymatic nucleic acid that is complementary to MIF or complementary to a MIF receptor, and combinations thereof.

3. The composition of claim 2, wherein the chemical compound comprising the isoxazoline moiety is p-hydroxyphenol-isoxazoline methyl ester or ISO-1.

4. The method of claim 1, wherein the bladder-related disorder is at least one of detrusor underactivity (DU), detrusor overactivity, detrusor hyperactivity, urinary retention, renal failure, or urinary tract infection.

5. The method of claim 1, wherein the antagonist or inhibitor comprises an antagonist of at least one of CD44, CD74 or both.

6. A composition for the treatment or prevention of detrusor underactivity comprising an effective amount of a chemical compound comprising an isoxazoline moiety or a pharmaceutically acceptable salt thereof, together with at least one of pharmaceutically acceptable carriers, excipients or adjuvants.

7. The composition of claim 6, wherein the composition further comprises at least one other biological agent for the treatment or prevention of DU.

8. The composition of claim 7, wherein the biological agent is at least one of bethanechol, an inhibitory nucleic acid, an enzymatic nucleic acid, an antibody or combinations thereof.

9. The composition of claim 6, wherein the chemical compound comprising the isoxazoline moiety is p-hydroxyphenol-isoxazoline methyl ester or ISO-1.

10. A isolated polynucleotide comprising a double stranded (ds) nucleic acid molecule that forms an siRNA and that down regulates expression of a MIF gene or MIF receptor gene via RNA-interference, wherein each strand of the ds nucleic acid molecule is independently about 10 to about 40 nucleotides in length; and wherein one strand of the ds nucleic acid molecule comprises a nucleotide sequence having sufficient complementarity to an RNA transcribed from the MIF gene or the MIF receptor gene for the ds nucleic acid molecule to cause, directly or indirectly, cleavage of said RNA via RNA-interference.

11. A method of diagnosing or monitoring a bladder-related disorder in a subject, comprising isolating DNA from a subject; and screening the DNA of a subject for at least one of polymorphism in a MIF gene or a MIF receptor gene, the expression level of a MIF gene or MIF receptor gene or both, wherein the gene comprises a nucleotide sequence of at least one of SEQ ID NO:2, 4, 6 or a combination thereof.

12. The method of claim 11, wherein said expression level is detected by measuring the RNA level expressed by said gene.

13. The method of claim 12, further comprising isolating mRNA from said patient prior to detecting the RNA level expressed by said gene.

14. The method of claim 11, wherein said polymorphism or expression level is detected by PCR or by hybridization to an oligonucleotide.

15. The method of claim 14, wherein the nucleotide sequence comprises DNA, RNA, cDNA, PNA, genomic DNA, or synthetic oligonucleotides.

16. The method of claim 11, wherein said expression is detecting by measuring levels of protein encoded by the gene.

17. The method of claim 14, wherein said polymorphism is present in 5′ UTR, 3′ UTR, intronic, or exonic DNA of said subject.

18. A method of treating or preventing a detrusor underactivity in a subject comprising the step of administering an effective amount of an antagonist or an inhibitor of MIF activity or of MIF receptor binding to an organism in need thereof.

19. The method of claim 18, wherein the antagonist or inhibitor is at least one member selected from the group consisting of a chemical compound comprising an isoxazoline moiety, an antagonist of a MIF receptor, an antibody to a MIF polypeptide; an antibody to a MIF receptor polypeptide, an enzymatic nucleic acid that is complementary to MIF or to a MIF receptor, and combinations thereof.

20. The composition of claim 19, wherein the chemical compound comprising the isoxazoline moiety is p-hydroxyphenol-isoxazoline methyl ester or ISO-1.
